(19) United States
(12) Patent Application Publication
(10) Pub. No.: US 2024/0228801 A9
(43) Pub. Date: Jul. 11, 2024

(54) SUPERHYDROPHOBIC ANTIFOULING COATING COMPOSITIONS AND APPLICATIONS THEREOF

(71) Applicant: University of Georgia Research Foundation, Inc., Athens, GA (US)

(72) Inventors: Hitesh HANDA, ATHENS, GA (US); Ekrem OZKAN, ATHENS, GA (US)

(21) Appl. No.: 18/546,207

(22) PCT Filed: Feb. 9, 2022
(86) PCT No.: PCT/US2022/015801

§ 371 Date: Aug. 11, 2023

Related U.S. Application Data
(60) Provisional application No. 63/148,945, filed on Feb. 12, 2021.

(51) Int. Cl. C09D 7/63; A01N 25/10; A01N 59/16; A01N 59/20; A01P 1/00; A61L 33/00; A61L 33/04; C09D 5/16; C09D 7/40; C09D 7/61; C09D 183/04

(52) U.S. Cl. CPC C09D 7/63; A01N 25/10; A01N 59/16; A01N 59/20; A01P 1/00; A61L 33/007; A61L 33/04; C09D 5/1618; C09D 5/1625; C09D 5/1681; C09D 7/61; C09D 7/67; C09D 183/04; A61L 2420/06

(57) ABSTRACT

Described herein are coating compositions having unique mechanical and physical properties. The coating compositions are composed of zinc oxide nanoparticles, copper nanoparticles, a perfluorolkylsiloxane, and an organic solvent. The coating compositions can be applied to any article or any surface of an article where it is desirable to reduce surface wettability or prevent the growth of bacteria.

### CROSS-REFERENCE TO RELATED APPLICATIONS

This application claims the benefit of and priority to co-pending U.S. Provisional Patent Application No. 63/148,945, filed on Feb. 12, 2021, the contents of which are incorporated by reference herein in their entireties.

### BACKGROUND

Biofilms can form on any surface exposed to environmental or physiological conditions during both short- and long-term operations. Free-floating (or planktonic) bacteria can come across a surface submerged in liquid and within minutes become attached. These free-floating bacteria are widely present and can find an easy way to the surface of the liquid-soaked material. The extracellular polymeric substances (EPSs) produced by the attached bacteria provide a source of nutrients for the stationary, attached bacteria. The evolving biofilm community is fed by the EPSs to mature into a complex, 3D biofilm structure. The biofilm protects the bacteria from the natural immune systems and antibiotics. Also, biofilms can survive against a broad range of treatments such as chlorine bleaching for 60 min and continuous flushing with multiple biocides over 7 days.

Biofilms pose serious threats to the function of a broad range of systems and devices including drinking water systems, plumbing, oil pipelines, and medical devices such as catheters, causing environmental, social, and economic implications. Therefore, strategies to prevent bacterial growth and biofilm formation on surfaces are of great interest, which either rely on chemical approaches that inactivate bacteria that do attach to the surface, that is, bactericidal activity, or physical approaches that inhibit initial bacterial attachment to the surface, that is, antibiofouling activity. The bacterial attachment on the surfaces is considered as the first step during biofilm formation. These needs and other needs are satisfied by the present disclosure.

### SUMMARY

Described herein are coating compositions having unique mechanical and physical properties. The coating compositions are composed of zinc oxide nanoparticles, copper nanoparticles, a perfluorolkylsiloxane, and an organic solvent. The coating compositions can be applied to any article or any surface of an article where it is desirable to reduce or prevent the development of biofilms.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1D provide SEM images of the surface morphologies of (a₁₋₃) original PU sponge and the painted PU sponges with (b₁-b₃) hydrophilic ZnO (H-PU-ZnO), (c₁-c₃) ZnO—Cu-10 (H-PU-ZnO—Cu-10), and (d₁-d₃) ZnO—Cu-20 (H-PU-ZnO—Cu-20) paints at different magnifications. The numbers refer to the weight percentage of the Cu NPs in the paints. (a₄-d₄) Optical photographs of methylene blue-dyed water droplets on the samples. For easy observation, water droplets were dyed with methylene blue.

FIGS. 2A-2E provide camera images of dyed water droplets on one layer of the NPs (a1-e1) and images of the NPs dispersed in water (a2-e2) or floating on the water surface without any wetting (a3-e3) because of their superhydrophobicity.

FIGS. 3A-3B provide images in accordance with embodiments of the present disclosure. (A) Images of vials containing superhydrophobic paints prepared from the ZnO/Cu mixtures with respect to Cu concentration (wt %) and (B) images of the original and treated PU surfaces with superhydrophobic FAS-ZnO, FAS-ZnO—Cu-10, and FAS-ZnO—Cu-20 paints, from left to right, followed by PDMS treatment. The numbers refer to the weight percentage of the Cu NPs in the paints. The dimensions of the samples are 6×2×2 cm.

FIGS. 4A-4F provide SEM images of the painted PU sponges with (a₁-a₂) superhydrophobic FAS-ZnO, (b₁-b₂) FAS-ZnO—Cu-10, and (c₁-c₂) FAS-ZnO—Cu-20 paints at different magnifications. The numbers refer to the weight percentage of the Cu NPs in the paints. (d₁-f₁) SEM images of the same samples after PDMS treatment. (d₂-₂) Expansion images from (d₁-f₁).

FIGS. 5A-5D are digital images of different liquid droplets for the as-prepared PU sponges. Liquids with a size of 20 μL on each sample are juice (yellow), milk (white), coffee (brown), and water (blue), respectively. (a2-d2) Photographs of the superhydrophobic sponges in water exhibiting a silver mirror-like water-sponge interface because of the existence of trapped air bubbles on the sponge surfaces unlike the original PU. (a3-d3) Images showing dyed-water droplets easily bouncing away after hitting the superhydrophobic sponges while the surface of the untreated sponge got contaminated.

FIGS. 6A-6C show the change in mouse fibroblast viable cells (as percentage relative to the control) (a) after 24 h [#: p<0.05 PU-ZnO—Cu-PDMS-10 vs PU-ZnO—Cu-PDMS-20. *: p<0.05 vs control, **: p<0.01 vs control], (b) after 24 h and 96 h exposure to various superhydrophobic sponge leachates [*: p<0.05 PU-ZnO—Cu-PDMS-10 (24 h leachate) vs 96 h leachate], and (c) after 24 h exposure to various hydrophilic sponge leachates [**p<0.01 vs control, ***p<0.001 vs control].

FIG. 7 shows inhibition of viable bacterial adhesion over 7 d exposure on the samples. (** indicates significance is p<0.01 compared to PU).

FIGS. 8A-8C show the degree of (a) fibrinogen and (b) platelet adhesion on various sponges. (* indicates significance is p<0.05 compared to PU).

FIGS. 9A-9D show (a) Pristine PU sponge and its blood contact angle, (b) PU-ZnO—Cu-PDMS-10 sponge and its blood contact angle, and (c) snapshots taken in the course of flowing blood on (c) pristine PU sponge and (d) PU-ZnO—Cu-PDMS-10 at a title angle of 10°.

FIGS. 10A-10L show durability tests for the PU-ZnO—Cu-PDMS-10 sample: (a-c) compression and recovery process of the sponge, (d,e) bending test, (f) tape-peeling test, (g) finger-wipe test, (h) single-hand-grasp, (i) both-hand-kneading, (j) knife scratch test, and (k,l) sandpaper test under a 250 g of loading weight on P400 sandpaper.

FIG. 11 shows the chemical structures of FAS-17 (on the left) and PDMS (on the right).

FIG. 12 shows a schematic illustration of the synthesis of FAS-grafted nanoparticles (FAS-ZnO and FAS-Cu NPs).

FIGS. 13A-13E show the FTIR analysis of (a) ZnO, (b) Cu, (c) FAS-ZnO, (d) FAS-ZnO—Cu-10 and (e) FAS-ZnO—Cu-20 powders.

FIG. 14 shows the SEM, EDS spectrum, TEM images, and XRD analysis of the untreated and treated powders.

FIGS. 15A-15B show the size distributions of (a) ZnO and (b) Cu NPs.

FIG. 16 shows the large size (27.5×14.5×2.5 cm) of the sponge painted by superhydrophobic FAS-ZnO—Cu-10 paint.

FIG. 17 shows the SEM and EDS mapping images of the PU-ZnO sponge.

FIG. 18 shows the SEM and EDS mapping images of the PU-ZnO-PDMS sponge.

FIG. 19 shows the SEM and EDS mapping images of the PU-ZnO—Cu-10 sponge.

FIG. 20 shows the SEM and EDS mapping images of the PU-ZnO—Cu-PDMS-10 sponge.

FIG. 21 shows the SEM and EDS mapping images of the PU-ZnO—Cu-20 sponge.

FIG. 22 shows the SEM and EDS mapping images of the PU-ZnO—Cu-PDMS-20 sponge.

FIG. 23 shows the contact angle of water and blood on various samples. Above: corresponding photographs of each liquid drop on the samples.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” “having,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a polysiloxane” includes, but is not limited to, mixtures or combinations of two or more such polysiloxanes, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “alkyl group” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl group has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, having from one to ten carbons, or from one to six carbon atoms in its backbone structure. Likewise, “lower alkenyl” and “lower alkynyl” have similar chain lengths. In embodiments described in the present application, preferred alkyl groups are lower alkyls. In some embodiments, a substituent designated herein as alkyl is a lower alkyl.

In some embodiments, a straight chain or branched chain alkyl group has 30 or fewer carbon atoms in its backbone (e.g., C₁-C₃₀ for straight chains, C₃-C₃₀ for branched chains), 20 or fewer, 12 or fewer, or 7 or fewer.

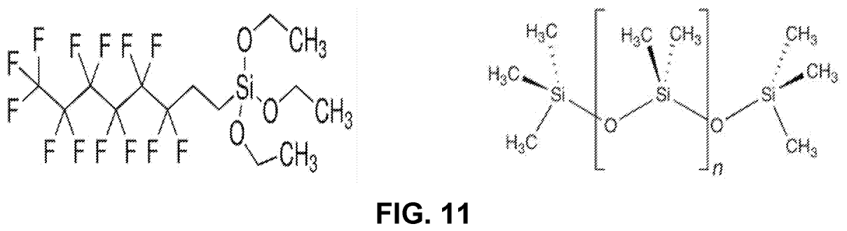

The term “perfluoroalkyl group” refers to an alkyl group as defined herein where two or more hydrogen atoms on the alkyl group are substituted with a fluorine atom. In one aspect, all of the hydrogen atoms on the alkyl group are substituted with a fluorine atom. FIG. 11 provides an exemplary structure of a perfluoroalkyl group bonded to a siloxane group (—Si(OEt)₃).

The term “prevent” or “preventing” as used herein is defined as eliminating or reducing the likelihood of the occurrence of one or more symptoms of a disease or disorder (e.g., biofilm formation) when using the compositions as described herein when compared to a control where the composition is not used.

### Coating Compositions and Applications Thereof

Described herein are coating compositions having unique mechanical and physical properties. The coating compositions are composed of zinc oxide nanoparticles, copper nanoparticles, a perfluorolkylsiloxane, and an organic solvent.

In one aspect, the zinc oxide nanoparticles can have an average particle size of about 20 nm to about 70 nm, or about 20 nm, 25 nm, 30 nm, 35 nm, 40 nm, 45 nm, 50 nm, 55 nm, 60 nm, 65 nm, or 70 nm, where any value can be a lower and upper endpoint of a range (e.g., 40 nm to 50 nm). In another aspect, The coating composition can include about 1% to about 20% zinc oxide nanoparticles by weight, or about 1%, 2%, 3%, 4%, 5%, 6%, 7%, 8%, 9%, 10%, 11%, 12%, 13%, 14%, 15%, 16%, 17%, 18%, 19%, or 20%, where any value can be a lower and upper endpoint of a range (e.g., 3% to 7%).

In one aspect, the copper nanoparticles can have an average particle size of about 20 nm to about 70 nm, or about 20 nm, 25 nm, 30 nm, 35 nm, 40 nm, 45 nm, 50 nm, 55 nm, 60 nm, 65 nm, or 70 nm, where any value can be a lower and upper endpoint of a range (e.g., 40 nm to 50 nm). In another aspect, the coating composition can include about 0.1% to about 5% zinc oxide nanoparticles by weight, or about 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, or 5.0%, where any value can be a lower and upper endpoint of a range (e.g., 0.3% to 1.5%).

The perfluorolkylsiloxane is a compound having a perfluoroalkyl group covalently bonded to a siloxane group. In one aspect, the perfluorolkylsiloxane can have the formula R²—Si(OR¹)₃, wherein R¹ is a substituted or unsubstituted C₁-C₂₀ alkyl group, and R² is a C₁-C₂₀ perfluoroalkyl group. In other aspects, R¹ can be a C₁ to C₄ alkyl group. R² can be a C₁ to C₁₀ perfluoroalkyl group. In one aspect, each R¹ can be methyl or ethyl, and R² can be a C₈ perfluoroalkyl group. In some embodiments, the coating composition can include about 0.1% to about 2% perfluorolkylsiloxane by weight, or about 0.1%, 0.2%, 0.3%, 0.4%, 0.5%, 0.6%, 0.7%, 0.8%, 0.9%, 1.1%, 1.2%, 1.3%, 1.4%, 1.5%, 1.6%, 1.7%, 1.8%, 1.9%, or 2.0%, where any value can be a lower and upper endpoint of a range (e.g., 0.3% to 1.5%).

In one aspect, the organic solvent can include an alcohol. In one aspect, the alcohol can be a C₁ to C₁₀ alcohol. In another aspect, the organic solvent can include, but is not limited to, methanol, ethanol, propanol, isopropanol, butanol, or any combination thereof. In another aspect, the organic solvent can be a hydrocarbon such as, for example, hexane. In one aspect, the coating composition can include about 73% to 98.8% of the organic solvent by weight, or about 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 98.8%, where any value can be a lower and upper endpoint of a range (e.g., 83% to 92%).

The coating compositions described herein can be made by admixing zinc oxide nanoparticles, copper nanoparticles, and a perfluorolkylsiloxane in an organic solvent. The components can be sequentially added to the organic solvent or, in the alternative, the components can be added concurrently to the organic solvent. Upon mixing of the components in the organic solvent, a homogeneous suspension is produced having a paint-like consistency. The duration of mixing of the components can vary as well as the temperature. In one aspect, the components are mixed at from 20° C. to 30° C., or at room temperature.

In one aspect, the weight ratio of the zinc oxide nanoparticles to the copper nanoparticles is from 1:1 to 20:1. In another aspect, the weight ratio of the zinc oxide nanoparticles to the copper nanoparticles is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1, where any value can be a lower and upper endpoint of a range (e.g., 5:1 to 10:1).

In one aspect, the weight ratio of the zinc oxide nanoparticles to the perfluoroalkysiloxane is from 5:1 to 20:1. In another aspect, the weight ratio of the zinc oxide nanoparticles to the perfluoroalkysiloxane is 5:1, 6:1, 7:1, 8:1, 9:1, 10:1, 11:1, 12:1, 13:1, 14:1, 15:1, 16:1, 17:1, 18:1, 19:1, or 20:1, where any value can be a lower and upper endpoint of a range (e.g., 10:1 to 15:1).

In one aspect, the weight ratio of the copper nanoparticles to the perfluoroalkysiloxane is from 0.5:1 to 5:1. In another aspect, the weight ratio of the copper nanoparticles to the perfluoroalkysiloxane is 0.5:1, 1:1, 1.5:1, 2:1, 2.5:1, 3:1, 3.5:1, 4:1, 4.5:1, or 5:1, where any value can be a lower and upper endpoint of a range (e.g., 1:1 to 2:1).

In one aspect, upon mixing the perfluorolkylsiloxane with the zinc oxide nanoparticles and the copper nanoparticles, the perfluorolkylsiloxane can form a covalent bond with the zinc oxide nanoparticles and/or the copper nanoparticles. In one aspect, the zinc oxide nanoparticles and the copper nanoparticles react with the siloxane group of the perfluorolkylsiloxane to produce new Si—Zn and Si—Cu bonds. In another aspect, the zinc oxide nanoparticles can have a hexagonal wurtzite structure and the copper nanoparticles can have a cubic structure.

Described herein are articles coated with the compositions described herein. In one aspect, the coated article is produced by (a) applying the coating composition as described herein to at least one surface of the article and (b) removing the organic solvent from the coating composition to produce the coated article. The coating composition can be applied to the article using techniques known in the art such as, for example, dipping or spraying. In one aspect, a single coating can be applied to the article. In other aspects, multiple coatings can be sequentially applied to the article. After the coating composition is applied to the article, the organic solvent is removed. In one aspect, the organic solvent can be removed by evaporation. In one aspect, the organic solvent can be removed by heating the coated article at a temperature of from about 80° C. to about 120° C. In one aspect, heating is sufficient to remove at least 80%, at least 85%, at least 90%, at least 95%, at least 99%, or 100% of the organic solvent. Upon heating in air, some of the copper nanoparticles may oxidize. In one aspect, the coating composition can also include Cu₂O after removal of the organic solvent.

After removing the organic solvent from the coating composition, in certain aspects, a polysiloxane can be applied to the coated article. The polysiloxane can include, but is not limited to, a polydimethylsiloxane, a polydiethylsiloxane, a polydipropylsiloxane, or a polydiphenylsiloxane. Not wishing to be bound by theory, the polysiloxane can improve the interaction of the zinc oxide nanoparticles and copper nanoparticles to the article. The polysiloxane can be formulated in a solvent such as, for example, chloroform, hexane, toluene, or dichloromethane. The polysiloxane solution can be applied to the article that has been previously coated with the coating composition. Thus, the polysiloxane is adjacent to (i.e., in intimate contact) with the zinc oxide nanoparticles and copper nanoparticles. In one aspect, the polysiloxane composition can include about 0.1% to about 5% zinc oxide nanoparticles by weight, or about 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 5.0%, 5.5%, 6.0%, 6.5%, 7.0%, 7.5%, 8.0%, 8.5%, 9.0%, 9.5%, or 10.0%, where any value can be a lower and upper endpoint of a range (e.g., 0.3% to 6.5%). The polysiloxane composition can be applied to the coated article using techniques known in the art such as, for example, dipping or spraying. After the polysiloxane composition has been applied, the coated article can be cured by heating the coated article.

The article to be coated can be any article or surface where it is desirable to reduce or prevent biofouling (e.g. growth of bacteria, adhesion of platelets, adhesion of fibrinogen). Biofilm and thrombus formation on surfaces results in significant morbidity and mortality worldwide, which highlights the importance of the development of efficacious fouling-prevention approaches. Provided herein are highly robust and superhydrophobic coatings with outstanding multi-liquid repellency, bactericidal performance, and extremely low bacterial and blood adhesion, which can be fabricated by a simple two-step dip-coating method.

In one aspect, the coating compositions described herein are useful in applications where it is desirable to reduce or prevent biofouling. Implantable medical devices are a leading cause of infection such as nosocomial infections. Implantable devices coated with or constructed with the compositions described herein can reduce or prevent biofouling in a subject when the device is introduced into the subject. In one aspect, the compositions described herein can reduce or prevent bacterial growth on a surface of an implantable device. In another aspect, the compositions described herein can reduce or prevent biofilm formation on a surface of an implantable device. In another aspect, the compositions described herein can reduce or prevent fibrinogen formation on a surface of an implantable device.

In one, the implantable device is a urinary catheter, artificial heart valve, a vascular catheter, a graft, or a stent. In other aspects, the device is intended to contact human blood or tissue. In one aspect, the device is a hemodialysis device or a component thereof. The coating compositions described herein are biocompatible (e.g., with fibroblast cells), which makes them useful in implantable medical devices.

In another aspect, the coating compositions described herein are useful in applications where it is desirable to reduce or prevent biofouling on polymeric medical grade materials (e.g. silicone, polyvinyl chloride (PVC), polyurethane (PU)). In other aspects, the coating compositions described herein are useful in applications where it is desirable to reduce or prevent biofouling on metals (e.g. steel, titanium). In other aspects, the coating compositions described herein are useful in applications where it is desirable to reduce or prevent biofouling on hospital touch surfaces (e.g. bed rails, bed frames, and handles).

In other aspects, the coating compositions described herein are useful in applications where it is desirable to reduce or prevent biofouling caused by the exposure to the environment. For example, the compositions described herein can be applied to any substrate that is exposed to environmental elements such as rain, snow, salt water, or other conditions that can cause or promote biofouling. In one aspect, the compositions described herein can be applied directly to the substrate using techniques known in the art such as, for example, spraying or dipping. In other aspect, the compositions described herein can be incorporated into a paint then subsequently applied to a substrate. In one aspect, the compositions described herein can be applied to automobile surfaces, boat hulls, or aircraft.

In one aspect, the coating compositions described herein can prevent the growth of bacteria on an article, in which the method includes applying the coating composition as above to at least one surface of the article. The coated article can prevent about at least 80%, at least 85%, at least 90%, at least 95%, at least 99%, or 100% of the growth of bacteria when compared to an uncoated article.

In one aspect, the coating compositions described herein can prevent the adhesion of fibrinogen on an article, in which the method includes applying the coating composition as above to at least one surface of the article. The coated article can prevent about at least 80%, at least 85%, at least 90%, at least 95%, at least 99%, or 100% of the adhesion of fibrinogen when compared to an uncoated article.

In one aspect, the coating compositions described herein can prevent the adhesion of platelets on an article, in which the method includes applying the coating composition as above to at least one surface of the article. The coated article can prevent about at least 80%, at least 85%, at least 90%, at least 95%, at least 99%, or 100% of the adhesion of platelets when compared to an uncoated article.

The coating compositions described herein are very hydrophobic (i.e., superhydrophobic). The degree of hydrophobicity can be measured by the contact angle of the coating. In one aspect, the coating and coated article has a receding contact angle of from about 140 degrees to about 175 degrees, or about 140 degrees, 145 degrees, 150 degrees, 155 degrees, 160 degrees, 165 degrees, 170 degrees, or 175 degrees, where any value can be a lower and upper endpoint of a range (e.g., 145 degrees to 160 degrees). In another aspect, the coating and coated article can have an advancing contact angle of from about 140 degrees to about 175 degrees, or about 140 degrees, 145 degrees, 150 degrees, 155 degrees, 160 degrees, 165 degrees, 170 degrees, or 175 degrees, where any value can be a lower and upper endpoint of a range (e.g., 145 degrees to 160 degrees). In another aspect, the coating and coated article can have a contact angle hysteresis of from about 0.1 degrees to about 5 degrees, or about 0.1 degrees, 0.5 degrees, 1.0 degrees, 1.5 degrees, 2.0 degrees, 2.5 degrees, 3.0 degrees, 3.5 degrees, 4.0 degrees, 4.5 degrees, or 5.0 degrees, where any value can be a lower and upper endpoint of a range (e.g., 1.5 degrees to 4.0 degrees). In another aspect, the coating and coated article can have a static water contact angle of greater than 150 degrees. With the high degree of hydrophobicity, the coating and coted articles described herein can repel various liquids including, but not limited to, water, milk, coffee, juice, and blood.

The coated articles described herein are capable of maintaining their unique physical and chemical properties (e.g., superhydrophobicity, anti-biofouling, etc.) when exposed to mechanical agitation. For example, the coatings are robust when subjected to different types of harsh mechanical agitation or damage such as, for example, finger-wiping, knife-scratching, tape-peeling, hand-kneading, hand-rubbing, bending, compress-release (1000 cycles) tests, and 1000 cm sandpaper abrasion under 250 g of loading.

### Aspects

Aspect 1. A coating composition comprising zinc oxide nanoparticles, copper nanoparticles, a perfluorolkylsiloxane, and an organic solvent.

Aspect 2. The composition of Aspect 1, wherein the zinc oxide nanoparticles have an average particle size of from about 20 nm to about 70 nm.

Aspect 3. The composition of Aspect 1 or 2, wherein the zinc oxide nanoparticles are from about 1% to about 20% by weight of the composition.

Aspect 4. The composition of any one of Aspects 1 to 3, wherein the copper nanoparticles have an average particle size of from about 20 nm to about 70 nm.

Aspect 5. The composition of any one of Aspects 1 to 4, wherein the copper nanoparticles are from about 0.1% to about 5% by weight of the composition.

Aspect 6. The composition of any one of Aspects 1 to 5, wherein the perfluorolkylsiloxane has the formula R²—Si(OR¹)₃, wherein R¹ is a substituted or unsubstituted C₂₀ alkyl group, and R² is a C₁-C₂₀ perfluoroalkyl group.

Aspect 7. The composition of Aspect 6, wherein R¹ is a C₁ to C₄ alkyl group.

Aspect 8. The composition of Aspect 6, wherein R² is a C₁ to C₁₀ perfluoroalkyl group.

Aspect 9. The composition of Aspect 6, wherein each R¹ is methyl or ethyl, and R² is a C₈ perfluoroalkyl group.

Aspect 10. The composition of any one of Aspects 1 to 9, wherein the perfluorolkylsiloxane is from about 0.1% to about 2% by weight of the composition.

Aspect 11. The composition of any one of Aspects 1 to 10, wherein the organic solvent comprises an alcohol or a hydrocarbon.

Aspect 12. The composition of any one of Aspects 1 to 10, wherein the organic solvent comprises a C₁ to C₁₀ alcohol.

Aspect 13. The composition of any one of Aspects 1 to 10, wherein the organic solvent comprises methanol, ethanol, propanol, isopropanol, butanol, or any combination thereof.

Aspect 14. The composition of any one of Aspects 1 to 13, wherein the organic solvent is from about 73% to about 98.8% by weight of the composition.

Aspect 15. The composition of any one of Aspects 1 to 13, wherein the composition is produced by mixing the zinc oxide nanoparticles, the copper nanoparticles, and the perfluorolkylsiloxane in the organic solvent.

Aspect 16. The composition of any one of Aspects 1 to 15, wherein the perfluorolkylsiloxane is covalently bonded to the zinc oxide nanoparticles and the copper nanoparticles.

Aspect 17. The composition of any one of Aspects 1 to 16, wherein the weight ratio of the zinc oxide nanoparticles to the copper nanoparticles is from 1:1 to 20:1, or is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1, where any value can be a lower and upper endpoint of a range (e.g., 5:1 to 10:1).

Aspect 18. The composition of any one of Aspects 1 to 17, wherein the weight ratio of the zinc oxide nanoparticles to the perfluoroalkysiloxane is from 5:1 to 20:1, or is 5:1, 6:1, 7:1, 8:1, 9:1, 10:1, 11:1, 12:1, 13:1, 14:1, 15:1, 16:1, 17:1, 18:1, 19:1, or 20:1, where any value can be a lower and upper endpoint of a range (e.g., 10:1 to 15:1).

Aspect 19. The composition of any one of Aspects 1 to 18, wherein the weight ratio of the copper nanoparticles to the perfluoroalkysiloxane is from 0.5:1 to 5:1, or is 0.5:1, 1:1, 1.5:1, 2:1, 2.5:1, 3:1, 3.5:1, 4:1, 4.5:1, or 5:1, where any value can be a lower and upper endpoint of a range (e.g., 1:1 to 2:1).

Aspect 20. A coated article produced by the method comprising (a) applying the coating composition of any one of Aspects 1 to 19 to at least one surface of the article and (b) removing the organic solvent from the coating composition to produce the coated article.

Aspect 21. The coated article of Aspect 20, wherein the article is dipped into the coating composition.

Aspect 22. The coated article of Aspect 20, wherein the coating composition is sprayed on at least one surface of the article.

Aspect 23. The coated article of any one of Aspects 20 to 22, wherein the organic solvent is removed by evaporation.

Aspect 24. The coated article of any one of Aspects 20 to 23, wherein step (b) comprises heating the coated article at a temperature of from about 80° C. to about 120° C. to remove the organic solvent.

Aspect 25. The coated article of any one of Aspects 20 to 24, wherein after step (b), applying to the coated article a polysiloxane.

Aspect 26. The coated article of Aspect 25, wherein the polysiloxane comprises a polydimethylsiloxane, a polydiethylsiloxane, a polydipropylsiloxane, or a polydiphenylsiloxane.

Aspect 27. A coated article comprising a first coating on at least one surface of the article, wherein the first coating comprises zinc oxide nanoparticles and copper nanoparticles covalently bonded to a perfluorolkylsiloxane.

Aspect 28. The coated article of Aspect 27, wherein the zinc oxide nanoparticles have a hexagonal wurtzite structure and the copper nanoparticles have a cubic structure.

Aspect 29. The coated article of Aspect 27 or 28, wherein the coating composition further comprises Cu₂O.

Aspect 30. The coated article of any one of Aspects 27 to 29, wherein the article further comprises a second coating comprising a polysiloxane adjacent to the first coating.

Aspect 31. The coated article of any one of Aspects 20 to 30, wherein the article comprises a polymeric grade material, a medical device, a surface or article in a hospital or medical facility, or a surface in an automobile, boat, or aircraft.

Aspect 32. The coated article of any one of Aspects 20 to 31, wherein the article has a receding contact angle of from about 140 degrees to about 175 degrees.

Aspect 33. The coated article of any one of Aspects 20 to 31, wherein the article has an advancing contact angle of from about 140 degrees to about 175 degrees.

Aspect 34. The coated article of any one of Aspects 20 to 31, wherein the article has a contact angle hysteresis of from about 0.1 degrees to about 5 degrees.

Aspect 35. The coated article of any one of Aspects 20 to 31, wherein the article has a static water contact angle of greater than 150 degrees.

Aspect 36. The coated article of any one of Aspects 20 to 31, wherein the article maintains superhydrophobic properties when exposed to mechanical agitation.

Aspect 37. The coated article of any one of Aspects 20 to 31, wherein the article is biocompatible.

Aspect 38. A method for preventing the growth of bacteria on an article, the method comprising applying the coating composition of any one of Aspects 1 to 19 to at least one surface of the article.

Aspect 39. A method for preventing the adhesion of fibrinogen on an article, the method comprising applying the coating composition of any one of Aspects 1 to 19 to at least one surface of the article.

Aspect 40. A method for preventing the adhesion of platelets on an article, the method comprising applying the coating composition of any one of Aspects 1 to 19 to at least one surface of the article.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Materials and Methods

Materials: Zinc oxide nanoparticles and copper nanoparticles were purchased from SkySpring Nanomaterials Inc. (Houston, USA). 1H,1H,2H,2H-Perfluorooctyltriethoxysilane was obtained from Oak-wood Chemical, Inc. (South Carolina, USA). Polyurethane sponge was obtained from a local store. MilliQ deionized (DI) water was utilized in all experiments; ethanol (200-Proof) was purchased from Decon Labs, Inc. (Pennsylvania, USA). PDMS (Sylgard 184) was purchased from Ellsworth Adhesives (USA). All chemicals were analytical-grade reagents and utilized without further purification.

Preparation of Superhydrophobic Coating Solutions and Particles: A total of 0.5 g of 1H,1H,2H,2H-perfluorooctyl-triethoxysilane (FAS-17) was placed into 50 g of absolute ethanol and magnetically stirred for 10 min at room temperature. Then, zinc oxide/copper NPs with different mass ratios were added to the abovementioned mixture (Table 1) and stirred continued for 10 min. Finally, the abovementioned mixtures were ultrasonicated for 5 min until homogeneous paint-like suspensions were formed. Through the evaporation of ethanol, FAS-treated hydrophobic particles were obtained. The particles prepared with only ZnO were denoted as FAS-ZnO, while those prepared with both ZnO and Cu were denoted as FAS-ZnO—Cu-10 and FAS-ZnO—Cu-20. The numbers refer to the weight percentage of the Cu NPs in the paints. Also, hydrophilic paints were prepared under the same conditions without the presence of FAS-17.

TABLE 1
Mixing Mass Ratio of the Precursors for Fabricating
Superhydrophobic and Hydrophilic Paints

| | ZnO NPs | Cu NPs | ethanol | FAS-17 |
|---|---|---|---|---|
| paint ID | (g) | (g) | (g) | (g) |
| superhydrophobic FAS-ZnO | 6 | | 50 | 0.5 |
| superhydrophobic | 6 | 0.6 | 50 | 0.5 |
| FAS-ZnO—Cu-10 | | | | |
| superhydrophobic | 6 | 1.2 | 50 | 0.5 |
| FAS-ZnO—Cu-20 | | | | |
| hydrophilic ZnO | 6 | | 50 | |
| hydrophilic ZnO—Cu-10 | 6 | 0.6 | 50 | |
| hydrophilic ZnO—Cu-20 | 6 | 1.2 | 50 | |

Preparation of Superhydrophobic Sponges. The as-prepared paint-like suspensions were deposited on commercial PU sponges (PU from hereon) by the dip-coating method (Table 2). Then, the sponges were dried at 100° C. for 1 h to obtain superhydrophobic samples. After manual squeezing to detach loose ZnO and Cu fragments from the sponges, the resulting sponges were dip-coated into a %0.5 PDMS-hexane solution, followed by curing to improve the weak interaction between the nanoparticles and sponges.

TABLE 2
Labeling of Superhydrophobic PU-ZnO and PU-ZnO—Cu Sponges
Depending on Concentration (wt %) and PDMS Coating

| | superhydrophobic | superhydrophobic | Superhydrophobic | |
|---|---|---|---|---|
| | FAS-ZnO | FAS-ZnO—Cu-10 | FAS-ZnO—Cu-20 | PDMS |
| sample ID | paint | paint | paint | coating |
| PU-ZnO | + | − | − | − |
| PU-ZnO-PDMS | + | − | − | + |
| PU-ZnO—Cu-10 | − | + | − | − |
| PU-ZnO—Cu-PDMS-10 | − | + | − | + |
| PU-ZnO—Cu-20 | − | − | + | − |
| PU-ZnO—Cu-PDMS-20 | − | − | + | + |

Instruments and Characterization. Scanning electron microscopy (SEM, FEI Teneo, FEI Co.) was performed at an accelerating voltage of 5.00 kV to examine the morphological features of the fabricated nanoparticles and sponges. Chemical microanalysis and elemental mapping were conducted with an energy dispersive X-ray spectroscopy (EDX, Oxford Instruments) system in which the microscope was equipped with an accelerating voltage of 20.00 kV. All samples were coated with 10 nm of gold-palladium with a Leica sputter coater prior to examination. The nanoparticles were imaged using a 100 kV JEOL JEM1011 transmission electron microscope (JEOL, Inc., Peabody, MA). The average diameter of the nano-particles was obtained using ImageJ software on the obtained images. Water contact angle (WCA) measurements were carried out using a DSA100 contact angle instrument (Germany). A 5-10 μL water droplet was placed on the surface of each sample and the final value was obtained by calculating the average of at least 5 measurements at different positions on each sample. Advancing (θ_{A}) and receding (θ_{R}) contact angles for each type of sample were taken to be average value of five measurements by adding and then withdrawing 5 μL of the water droplet, respectively, on each sample surface. The contact angle hysteresis (θ-θ) was obtained for each sample. Chemical changes of the particles were investigated via Fourier-transform infrared (FTIR) spectroscopy using a Thermo Fisher Nicolet 6700 spectrometer (Waltman, MA) with a Harrick VariGATR grazing angle ATR accessory (Pleasantville, New York). Dry powder samples were measured over a range of 600-4000 cm⁻¹ with 128 scans performed at a resolution of 4 cm⁻¹ per sample. Purity and crystalline nature of the samples were studied by X-ray diffraction (XRD) spectroscopy with a PANalytical X'pert PRO MRD diffractometer equipped with CuKα1 radiation (λ=1.541 A) in the 2θ range from 10° to 80° with a scanning rate of 0.03° s⁻¹.

Preparation of Leachates. The samples were soaked in Dulbecco's modified Eagle medium (DMEM) for 4 days in 37° C. and the solutions were then used for measuring metal nanoparticle (ZnO and Cu) leachates using a VG ICP-MS Plasma Quad 3 instrument. The samples were removed from the media before the media were analyzed for the presence of ⁶⁵Cu and ⁶⁶Zn according to a previously published method.^{40,41}

In Vitro Cytotoxicity Assay. WST-8 based Cell Counting Kit-8 (CCK-8) assay was used to measure the cell viability of superhydrophobic and hydrophilic sponges on NIH 3T3 mouse fibroblast cells (ATCC 1658). NIH 3T3 cells (5000 cells/mL) were cultured in DMEM containing 4.5 g L⁻¹ glucose and L-glutamine, 10% FBS, and 1% penicillin-streptomycin at 37° C. under a humidified atmosphere with 5% CO₂ in 96-well plates. Leachates from superhydrophobic and hydrophilic sponges were prepared by soaking the samples in DMEM (1 mL of medium per 1 mg of sample) and incubated at 37° C. for 24 and 96 h for some superhydrophobic sponges. After 24 h, the leachates were added to each well and incubated for another 24 h. The change in the number of viable cells of NIH 3T3 cells due to possible toxic leachates from sponge samples was assessed by adding 10 μL of CCK-8 solution to each well after 24 h, followed by incubation for 3 h at 37° C. The absorbance at 450 nm was determined using a multiplate reader (Biotek Cytation 5). A comparison was made between untreated cells (control) and viable cells in the presence of leachates and reported as percentage relative to control.

In Vitro Bacterial Adhesion and Growth in a Drip-Flow Bioreactor Model. The prevention of bacterial adhesion on the designed antimicrobial sponges was studied using a 4 d drip-flow bioreactor model. To obtain the required pathogenic culture, a colony of Staphylococcus aureus was inoculated in Luria-Bertani (LB) broth media and grown overnight to a CFU mL⁻¹ of 10⁶ to 10⁸. The overnight culture was then centrifuged to obtain a bacterial pellet which was then washed in phosphate-buffered saline (PBS) by centrifuging at 4400 rpm for 7.5 min. Washing with PBS is done to discard any waste (supernatant), and the pellet obtained is then resuspended in PBS to obtain a CFU of 10⁶ to 10⁸ mL⁻¹.

While the bacterial suspension culture for the incubation period is prepared, simultaneously, the drip-flow bioreactor (Biosurface Technologies, DFR) is prepared for the study by autoclaving it and connecting it to the required components (2 g L⁻¹ LB broth, peristaltic pump set at 0.8 mL min⁻¹). A revised form of the ASTM E2647-13 protocol was used for the experiment. The samples to be tested were placed in the chambers of the sterile DFR and incubated with the prepared bacterial solution (10⁶-10⁸ CFU mL⁻¹) for 4 h. This 4 h incubation was done to allow for the S. aureus bacteria to settle on the sponge surface. After 4 h of incubation, 2 g L⁻¹ LB broth media was flowed through the chambers at a rate of 0.8 mL min⁻¹. This flow rate was used to allow for low shear conditions and a more rigorous test for antimicrobial efficacy. At the end of 4 d, the samples were rinsed to remove any unadhered bacteria on the surfaces. Following this, the samples were homogenized to remove the adhered bacteria and the obtained bacterial samples were serially diluted. The serial dilutions were then plated, and colonies were counted after 18 h of incubation in 37° C.

The following formula was used for the bacterial calculations:

CFU of S. aureus cm⁻² of the sample

$$total CFU = \frac{CFU \times dilution factor \times suspension in solution}{suspension plated}$$

$$CFU of S . aureus {cm}^{- 2} of sample = \frac{total CFU}{surface area of sample}$$

Statistical Analysis. Data for the 4 d bacterial adhesion analysis is expressed as mean±standard deviation (SD). The results between the data for the sponges were analyzed by comparison of means using Student's t-test. Values of p<0.05 were considered statistically significant for all tests.

Adsorption of Fibrinogen In Vitro. Levels of protein adhesion were quantified for the fabricated sponge coatings using a modified version of a previously reported method.42 FITC-labeled human fibrinogen was diluted with unlabeled fibrinogen solution to achieve a 1:10 ratio of 4 mg mL-1 fibrinogen in phosphate buffer solution (pH 7.4). Sections of the various sponge samples were incubated in phosphate buffer solution at 37° C. for 30 min in a 96-well plate, followed by the addition of the physiological fibrinogen solution to achieve a final concentration of 2 mg mL-1. After 90 min of incubation in the protein solution, samples were infinitely diluted in order to wash away any loosely bound protein from the sponges. Adsorbed fibrinogen was quantified by measuring the excitation/emission of sample at 495/519 nm and interpolated using a standard curve of FITC-labeled fibrinogen with a 1:10 dilution factor.

Assessment of Platelet Adhesion In Vitro. Samples were exposed to blood plasma with a known quantity of platelets in order to assess antiplatelet efficacy. All protocols pertaining to the use of whole blood and platelets were approved by the Institutional Animal Care and Use Committee. Freshly drawn porcine blood (Lampire Biological) with 3.9% sodium citrate at a ratio of 9:1 (blood/citrate) was used. The anticoagulated blood was centrifuged at 300 rcf for 12 min using a Beckman Coulter Allegra X-30R centrifuge. The platelet-rich plasma (PRP) portion was collected carefully with a pipet so as to not disturb the buffy coat. The remaining samples were then spun again at 4000 rcf for 20 min to collect platelet-poor plasma (PPP). Total platelet counts of both the PRP and PPP fractions were determined using a hemocytometer (Fisher). The PRP and PPP were combined in a ratio to give a final platelet concentration 2×10⁸ platelets mL⁻¹. Calcium chloride (CaCl₂) was added to the final platelet solution to reverse the anticoagulant (Na-citrate), and thereafter, samples were placed in blood tubes and exposed to approximately 4 mL of the calcified PRP. The tubes were then incubated at 37° C. for 90 min with mild rocking (25 rpm) on a Medicus Health blood tube rocker. Following the incubation, the tubes were infinitely diluted with 0.9% saline solution. The degree of platelet adhesion was determined using the lactate dehydrogenase (LDH) released when the adherent platelets were lysed with a Triton-PBS buffer (2% v/v Triton-X-100 in PBS) using a Roche cytotoxicity detection kit (LDH). A calibration curve was constructed using known dilutions of the final PRP solution, and the platelet adhesion of the various sponge types was interpolated from the calibration curve.

Different Mechanical Tests. Various frictions tests including finger-wipe, single-hand-grasp, and both-hand-kneading tests have been performed using fingers, followed by water dropping onto the sample after each test. For the bending test, the sponge was repeatedly bent forward and backward, from −90 to 90° (defined as 1 cycle) over 100 folding cycles, and then, water droplets were dropped on the sample. For the tape-peeling test, the sponge was pressed by 250 g loading with adhesive tape, and then, the tape was peeled off trying to remove the superhydrophobic coating from the sponge surface. This process was defined as 1 cycle and repeated at least 100 times. Additionally, the sample was manually compressed to over 50% and released (defined as 1 cycle). This process was repeated 1000 times and then water droplets were dropped onto the sample. To further show the mechanical stability, the sample with a weight of 250 g was placed above it facedown onto the sandpaper (standard glasspaper, grit no. 400) and was longitudinally and transversely rubbed for over 1000 cm. Then, water droplets were dropped on the sample. Also, the sponge surface was scratched with a scalpel in air, and subsequently, water droplets were dropped onto the sample.

### Results and Discussion

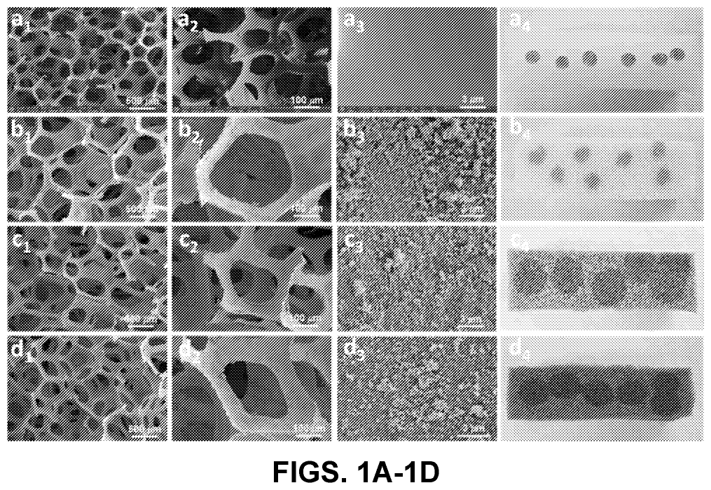

SEM was used to characterize the surface evolution of the sponges before and after the dip-coating process using hydrophilic paints without FAS. As demonstrated in FIG. 1a, the plain sponge was hydrophobic in nature (WCA ˜113°) and possessed a very smooth, interconnected three-dimensional (3D) framework with an average pore size ranging from tens to several hundred micrometres. However, although plenty of the bare NPs generated rough hierarchical topo-graphical structures on the surfaces of the originally smooth sponges (FIG. 1b-d), they could not endow the sponges with superhydrophobicity. On the contrary, the sponges loaded with the bare NPs could quickly absorb water droplets because of the hydrophilic nature of the NPs (FIG. 1b₄-d₄, indicating the key role of low surface free energy in achieving the nonwetting property.

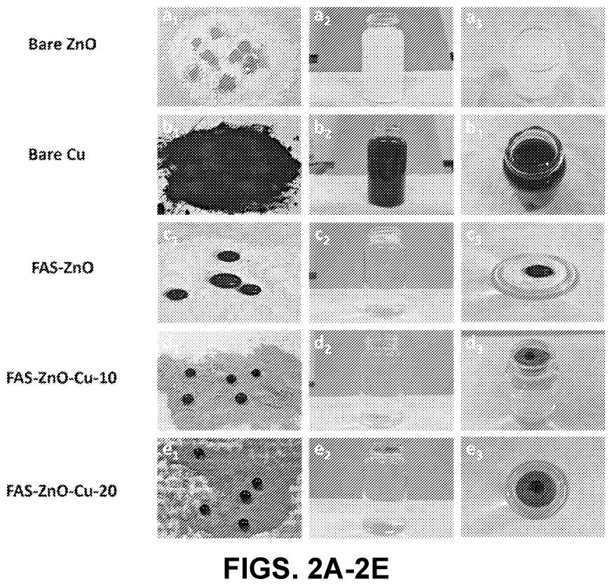

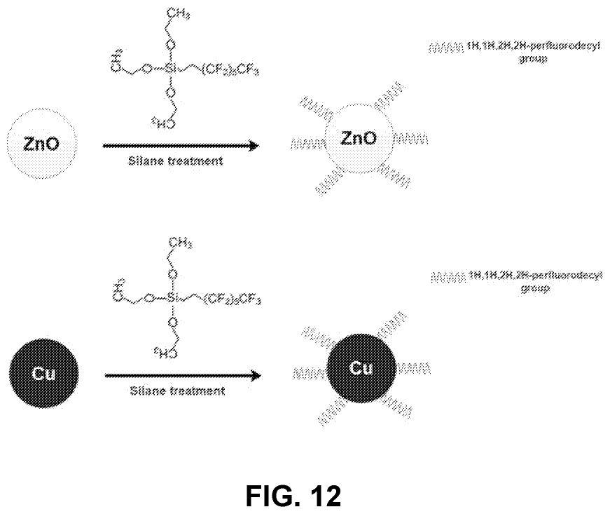

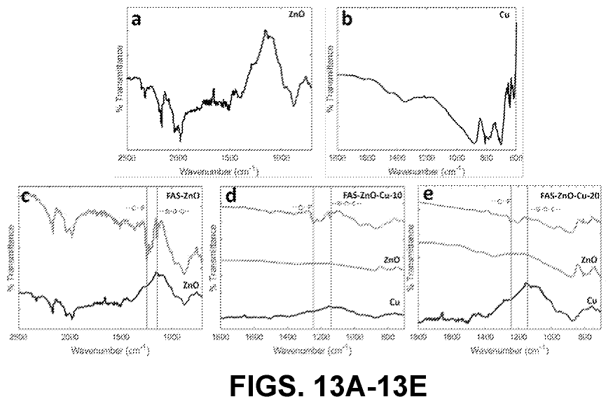

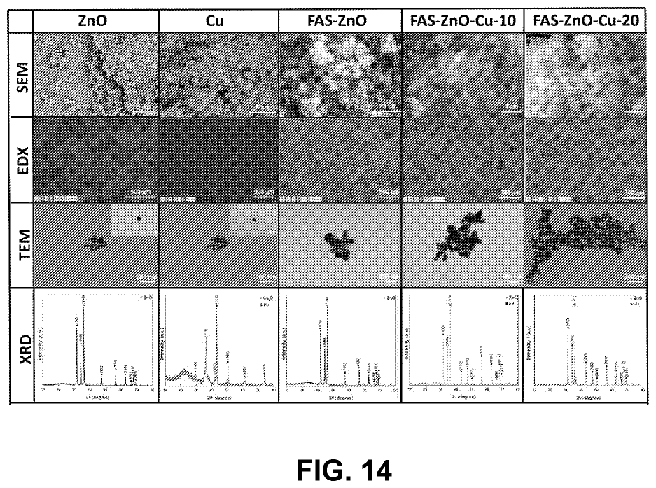

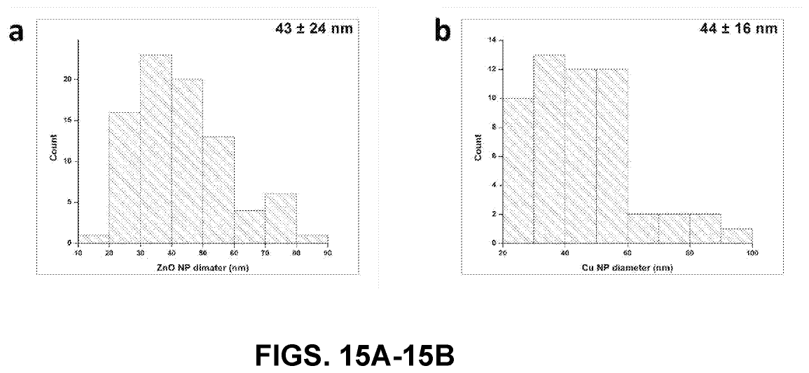

However, after treatment with FAS-ethanol solutions, FAS molecules can covalently attached to the surface of the NPs (FIG. 12), changing the surface wettability of the particles from superhydrophilic to superhydrophobic by reducing their surface free energy. This is in good agreement with the FTIR analysis, revealing that after FAS modification, compared to the bare NPs, two new absorption peaks at the wavenumber of 1145 and 1250 cm⁻¹ appeared (FIG. 13), which are ascribed to Si—O—C and C—F stretching of the FAS molecules in accordance with previous literature.⁴⁴ This confirms that low-surface-energy FAS molecules have been successfully anchored on the surface of the NPs. The existence of FAS molecules on the NPs was also proved by the exceptional increase in their wetting resistance. As it can be seen from FIG. 2a₁-b₁, when the testing liquid droplets were dropped onto the native nanoparticles, they were quickly absorbed by the particles. However, all FAS-coated NPs showed super-hydrophobicity as demonstrated in FIG. 2c₁-e₁; the dripped droplets remained as near spheres and could easily roll off from the surfaces without any wetting. Also, when sprinkled in water, the treaded nanoparticles stay afloat on water without any wetting for at least 3 months (FIG. 2c₂-e₂), while the untreated NPs were completely mixed with deionized (DI) water (FIG. 2a₂,b₂). More detailed characterization (e.g., SEM, EDX, TEM, and XRD) of the fabricated particles is demonstrated in FIG. 14-15.

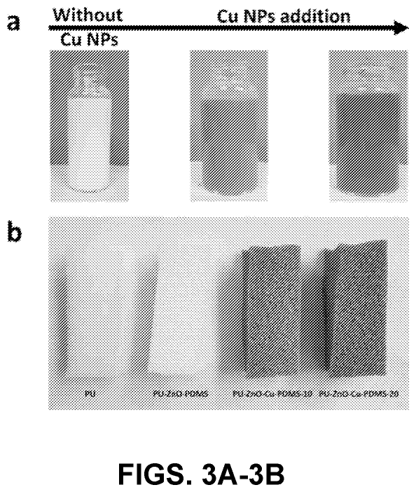

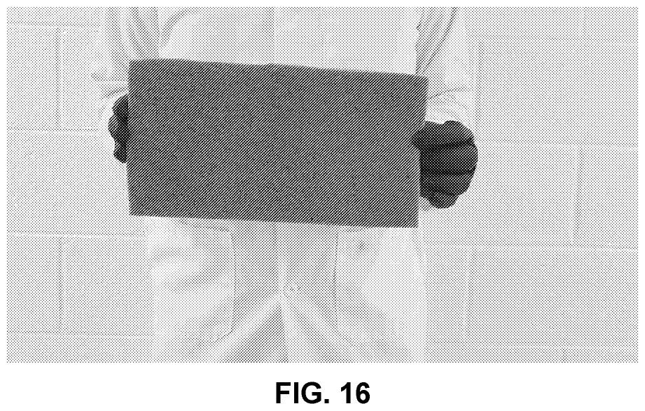

The treated particles can be used to render surfaces superhydrophobic, originating from their hydrophobic nature and intrinsic micro/nanostructure. In a typical preparation process, three individual superhydrophobic paint-like solutions made of ZnO/Cu NPs with different Cu weight ratios and a low surface modifier, FAS-17, in ethanol were prepared. The paint color turned from white to gray by adding Cu NPs into the solution and the color became more intense with increasing Cu concentration (FIG. 3a). Then, the paint-like solutions were applied onto commercial PU sponges through dip-coating. Finally, the modified sponges were dip-coated with PDMS-hexane solution to strengthen the bonding degree between the NPs and the sponge surfaces. FIG. 3b demonstrates the digital photograph of the obtained sponges after dip-coating treatments. The PU sponge colour changed from white to yellowish-white when exposed to just ZnO NPs, whereas those exposed to both ZnO and Cu NPs varies from gray to dark gray depending on the concentration of Cu NPs in the paints. Additionally, the dip-coating method can be used to apply the liquid-repellent coatings to large-scale fabrication (FIG. 16).

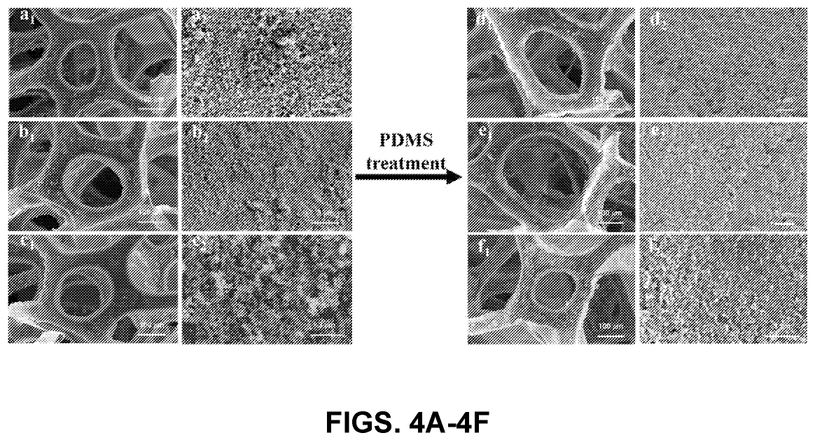

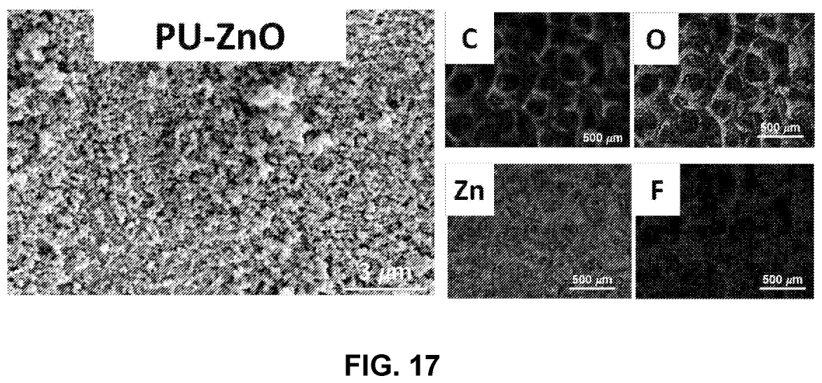

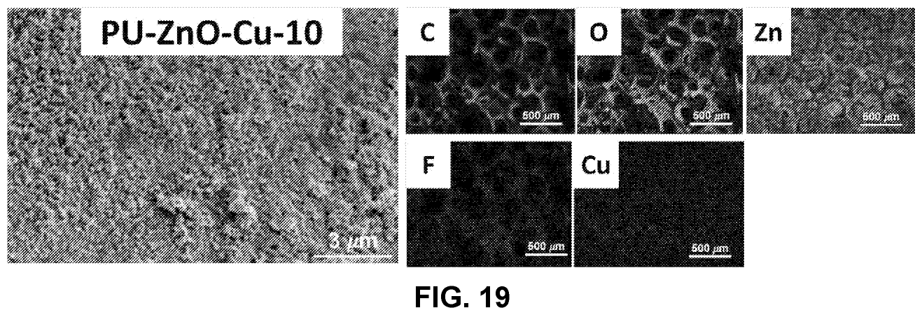

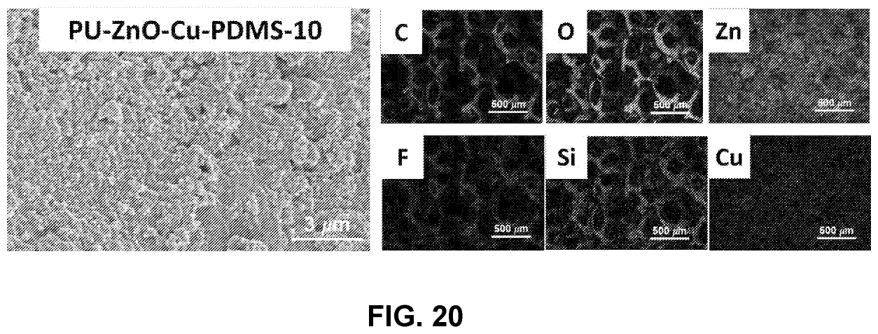

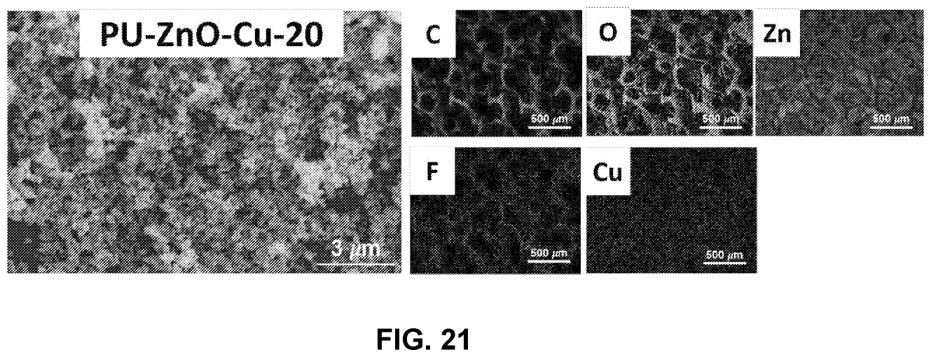

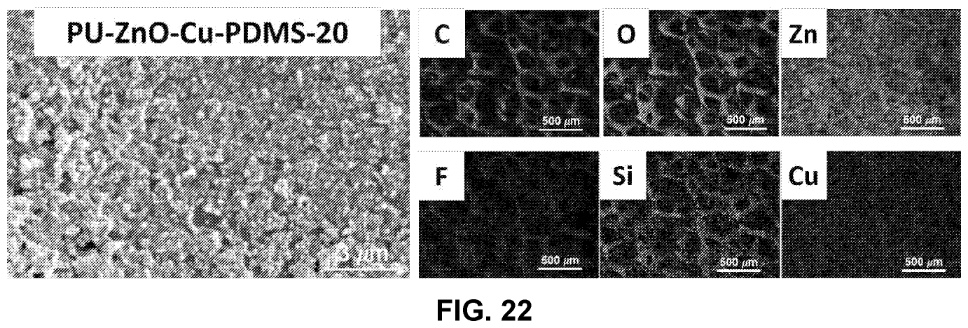

The surface morphologies of the original and treated hydrophobic NP-coated PU sponges before and after PDMS coating were characterized by SEM (FIG. 4). It is evident that while the coating treatment preserved their original structure without blocking the interconnected pores of the 3D sponges, rough hierarchical micro- and nanoscale structures were formed on the surface of the PU scaffolds by assembly of superhydrophobic ZnO and Cu particles (FIG. 4a-c). Post-PDMS coating resulted in a homogeneous particle packing structure with PDMS as particle interconnection covering the particles (FIG. 4d-f). As demonstrated in EDX analysis in FIGS. 17-S12, the treated sponges contain the F element, which further confirms that the sponge skeletons were successively coated with FAS-treated nanoparticles. The post-PDMS coating caused an increase in the distribution of the Si element in the samples. Moreover, increasing incorporated concentrations of Cu were confirmed through Cu mapping between both ZnO—Cu sponges (FIGS. 19 and 21) and ZnO—Cu-PDMS sponges (FIGS. 20 and 22) and the composition of Zn, C, and O was relatively consistent between each sample.

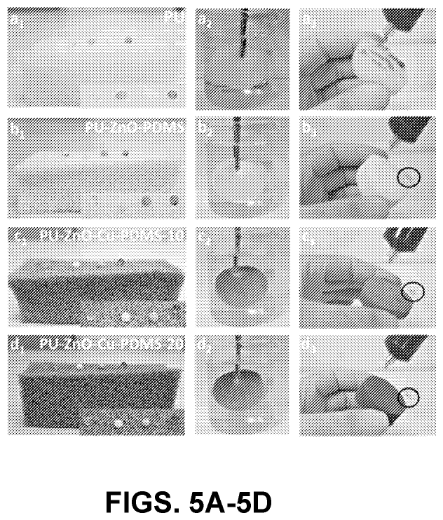

The treated sponges with the hierarchical topographical textured structures with low surface energy possess an exceptional ability to trap air and hence construct surface solid-liquid-air interfaces, resulting in a prominent liquid repellence that forces a broad range of liquids to resemble typical spherical balls on all the coated substrates, enabling them to readily slide off without leaving any traces (FIG. 5b₁-d₁). On the other hand, as demonstrated in FIG. 5a₁, the bare sponge can be wetted by all tested liquid droplets that would either completely spread or show semispherical shapes on the bare sponge, presenting poor liquid repellent properties, and there was much adhesion apparent as the droplets slid on the included bare sponge, indicating the strong adhesion of water droplets to the surface, which is further indicated by the large roll-off angle (more than 90°).

To further examine the remarkable water repellency of the treated sponges, they were completely submerged in water by an external force. Unlike the bare sponge (FIG. 5a₂), the superhydrophobic sponges (FIG. 5b₂-d₂) demonstrated an obvious bright plastron layer (i.e., a layer of air) because of the total reflectance of light at the air layer trapped on the surface, preventing the sponges from wetting, and they would instantaneously refloat after the force is withdrawn and stay completely dry. Water droplets can also bounce off the coatings without leaving any trace because of the weak water-surface interaction, indicating very low contact angle hysteresis (<5°, Table 5) that confirms uniformity and lack of pinning points (FIG. 5b₃-d₃).⁴⁵ On the contrary, the water droplets caused a flat puddle on the naive sponge surface because of the strong water-surface interaction and could fully wet the surface, indicating high contact angle hysteresis (FIG. 5a₃).

TABLE 5
Advancing Contact angle, Receding Contact angle,
and Contact angle Hysteresis for various sponges

| | advancing | receding | |
|---|---|---|---|
| | contact | contact | contact angle |
| sample | angle (°) | angle (°) | hysteresis (°) |
| PU | 109.6 ± 2.1 | 69.6 ± 2.5 | 40.0 ± 2.7 |
| PU-ZNO-PDMS | 157.5 ± 2.8 | 156.4 ± 2.9 | 1.11 ± 0.9 |
| PU-ZnO—Cu-PDMS-10 | 157.7 ± 2.1 | 156.6 ± 2.1 | 1.10 ± 0.6 |
| PU-ZnO—Cu-PDMS-20 | 161.6 ± 1.0 | 160.8 ± 0.9 | 0.8 ± 0.2 |

Detection of Zinc and Copper Leaching. Before performing any in vitro biological assays, it is necessary to measure the NPs leachates from the materials fabricated as a high leaching of NPs can set off cytotoxic and inflammatory actions.^{46,47} Thus, it is important to maintain just enough metal NP release to function as a bactericidal material without causing cytotoxic damage to mammalian cells. To estimate the ZnO-NP and Cu-NP diffusion into the physiological environments, ICP-MS analysis was performed on DMEM that was exposed to the samples for 4 days (Table 3).

TABLE 3
Zinc and Copper Leachate Concentration
from the Various Samples in DMEM (n = 4)

| sample | ⁶⁵Cu (μg/mL) | ⁶⁶Zn (μg/mL) |
|---|---|---|
| PU-ZnO-PDMS | | 5.392 ± 2.911 |
| PU-ZnO—Cu-PDMS-10 | 3.283 ± 0.900 | 6.681 ± 2.361 |
| hydrophilic PU-ZnO hydrophilic | | 12.472 ± 1.285 |
| PU-ZnO—Cu-10 | | |
| | 26.412 ± 0.972 | 8.439 ± 2.299 |

As mentioned in the literature earlier, we know that 10 μg/mL of Zn can cause DNA damage in nasal mucosal cells, and thus, keeping that mind,⁴⁶ we can assume that the leaching of Zn from the sponges will not cause any cytotoxicity except from the hydrophilic samples. From the literature, it is also known that at a concentration of 25-50 μg/mL Cu NPs can be cytotoxic to mammalian cells. ⁴⁸⁻⁵⁰ Although the PU-ZnO—Cu-PDMS-10 sponge maintains a low leaching of 3.283±0.900 μg/mL, the hydrophilic sponges leach a considerable amount of Cu which can be potentially cytotoxic. The minimal leaching of Zn and Cu within the PDMS matrix in the superhydrophobic surface demonstrates the potential longevity of their bactericidal activity. This slow rate of release would ensure bactericidal activity for long-term applications and thus help in maintaining the uncontaminated surface of the antifouling superhydrophobic surface. The balance of Zn and Cu release from the polymer matrix ensures minimal cytotoxic activity and also maintains the bactericidal nature of the surface, as discussed later.

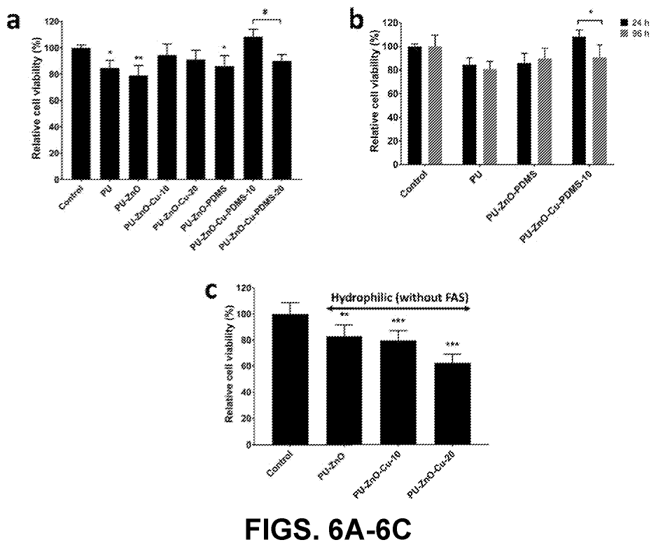

Cytocompatibility of the Sponges. A major aspect in biocompatibility evaluations for potential biomedical application includes evaluation of toxicity elicited by the material toward mammalian cells in vitro. Cytotoxicity was evaluated based on the ISO 10993 protocol using a WST-8 dye-based CCK-8 assay. The CCK-8 assay was used to measure the conversion of a highly water-soluble tetrazolium salt, 2-(2-methoxy-4-nitrophenyl)-3-(4-nitrophenyl)-5-(2,4-di-sulfophenyl)-2H-tetrazolium, monosodium salt (WST-8), to a water-soluble formazan dye upon reduction by dehydrogenases in the presence of an electron carrier in living cells. The change is detectable spectrophotometrically at 450 nm. 3T3 mouse fibroblast cells were employed for the study as it is an established model cell line for various cell response studies.^{51,52} Initially, superhydrophobic sponges containing different concentrations of ZnO and Cu NPs as constituents of the sponges were evaluated for 24 h cytotoxicity, as shown in FIG. 6a. Based on the cytotoxicity evaluations, PU-ZnO-PDMS and PU-ZnO—Cu-PDMS-10 were chosen for further cytotoxicity assessment with 96 h of the leachate collected, as seen in FIG. 6b. Interestingly, when 3T3 cells were exposed to PU-ZnO—Cu-PDMS-10 leachates, the number of viable cells slightly increased. This slight increase (p<0.05) can be attributed to a slow and stabilized leaching of Cu NPs due to the presence of a PDMS layer. Owing to the stabilizing effect of PDMS and superhydrophobicity of the sponge, slow release of Cu NPs affects the cell viability in a positive manner. Studies have demonstrated that the risk of Cu toxicity can be reduced by significantly slowing down the leaching of Cu.^{53,54} Previous studies have shown that both Cu and ZnO NPs are not cytotoxic at very low concentrations.⁴⁸⁻⁵⁰ Results from ICP-MS show very low leaching concentrations for both Cu and ZnO, which explains the noncytotoxicity of the leachates. The slow release can, in principle, promote cell migration and improve the wound-healing process⁵⁵ and stimulate angio-genesis,⁵⁶ which requires cell proliferation. In wound healing, Cu^{2 +} stimulates the expression of matrix metalloproteinase-2 and collagen in fibroblasts.⁵⁷ However, when the Cu NP concentration in the polymer composite was doubled, it showed significant reduction in the number of viable 3T3 cells.

For hydrophilic sponges, the rate of leaching increases because of the availability of water and lack of PDMS coating to stabilize the release of leachates. As a result, a decrease in the number of viable 3T3 cells was observed with increasing Cu concentration on the hydrophilic sponges. There is a statistically significant trend of a decreasing number of viable cells, as seen in FIG. 6c. Cao et al. have demonstrated a considerable amount of cytotoxicity at 25-50 μg/mL of Cu ion concentration on L929 mouse fibroblast cells.⁵⁸ The toxic effect elicited by Cu NPs is mainly attributed to cell cycle arrest. ZnO, in this case, did not have a significantly greater amount of leaching than its superhydrophobic counterpart. Thus, reduction in the number of viable cells for hydrophilic sponges could potentially be due to Cu NP leaching. The cellular responses can, however, potentially vary for different cell lines. For example, Saranya et al. found that synthesized copper powders demonstrated varying cytotoxic effects on Vero, PK 15, and MDBK cells.⁴⁸ The current study can be the basis of further evaluation of the material cytocompatibility.

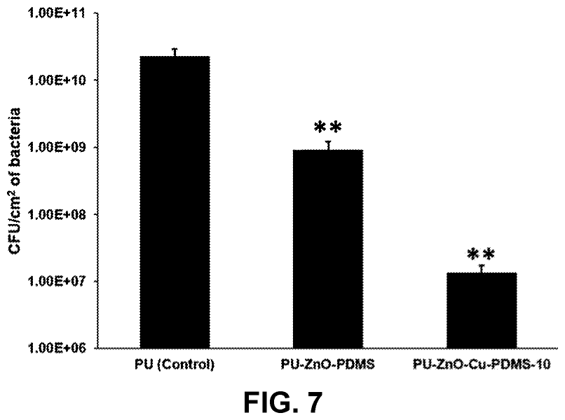

Bacterial Adhesion on the Samples after 4 d of Exposure. The superhydrophobic antimicrobial sponges' ability to inhibit bacterial infection by testing them in a 4 d drip-flow bioreactor model was examined. Based on the cytotoxicity evaluations, the following samples were chosen to evaluate their antimicrobial activity against the Gram-positive bacteria S. aureus:⁵⁹ PU sponge (control), PU-ZnO-PDMS, and PU-ZnO—Cu-PDMS-10. Drip-flow bioreactor models have been used previously to study biofilm formation for developing antimicrobial materials.⁶⁰ Because biofilms tend to grow better in a drip-flow system, compared to a CDC high-shear bioreactor,⁶¹ the antimicrobial efficacy of the superhydrophobic sponges would have to be high to reduce the adhesion of bacteria. As seen in FIG. 7 (n=5), the bacterial adhesion on PU-ZnO-PDMS and PU-ZnO—Cu-PDMS-10 samples was reduced by 95.99±1.36% (p=0.001) and 99.94±0.02% (p=0.001), respectively, when compared to PU sponge control samples, indicating that they possess long-term antifouling properties. Additionally, the bacterial adhesion on PU-ZnO—Cu-PDMS-10 samples was reduced by 98.55±0.43% (p=0.004) when compared to PU-ZnO-PDMS samples (Table 4). As hypothesized previously, the bacterial adhesion reduction was greater in PU-ZnO—Cu-PDMS-10 samples, possibly because of the addition of antimicrobial Cu NPs to the ZnO-NP coated sponge samples. In previous studies done by our group, we have been able to separately show the antimicrobial effects of both Cu and ZnO-NPs, but in dip-coated polymer device coatings.^{40,41} The antimicrobial effects of the metal nanoparticles are further enhanced by the superhydrophobic nature of the foam samples which can prevent the attachment of EPSs to form mature biofilms. In general, the resistance to bacterial adhesion may be ascribed to the liquid-repelling nature of the treated sponges, which inhibited the access of the organisms to the nutrients and moisture required for growth.

TABLE 4
Comparative Analysis of Bacterial
CFU/cm² Grown on the Samples

| | PU | PU-ZnO- | PU-ZnO—Cu- |
|---|---|---|---|
| | (control) | PDMS | PDMS-10 |
| CFU cm⁻² of S. aureus | 2.299 × 10¹⁰ | 9.205 × 10⁸ | 1.330 × 10⁷ |
| reduction compared to PU | | 95.99 ± 1.36 | |
| (control) (%) | | | 99.94 ± 0.02 |
| reduction compared to | | | |
| PU-ZnO-PDMS (%) | | | 98.55 ± 0.43 |
| p value vs PU (control) | | 0.001 | 0.001 |
| p value vs PU-ZnO-PDMS | 0.001 | 0.004 | |

To validate the antimicrobial effectiveness of these super-hydrophobic surfaces, their antimicrobial activity was compared to other superhydrophobic surfaces published in literature. It should be noted most studies have examined the antimicrobial properties of superhydrophobic surfaces for relatively short time scales. Following 4 h of exposure to E. coli suspension, the surfaces exhibited a 3.2 log reduction in the adhesion of bacteria compared with bare glass. It was found that after 5 days of incubation under static conditions, biofilm formation was more extensive on the superhydrophobic surface compared to the PDMS surface.⁶⁵ Their results emphasize the excellent long-term antimicrobial activity exhibited by the PDMS-ZnO—Cu-PDMS sponges reported here.

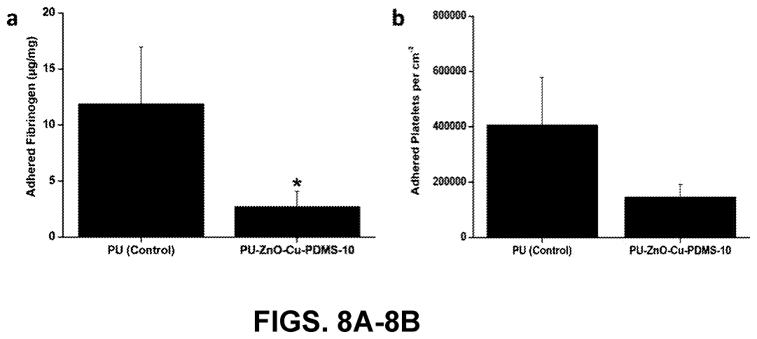

Reduced Adhesion of Blood Components. Adhesion and activation of fibrinogen and platelets, two major blood components, cause surface-induced thrombosis.⁶⁶ Therefore, we exposed the original and PU-ZnO—Cu-PDMS-10 sponges to a fibrinogen solution at physiological concentration for 90 min. Because the majority of blood proteins adsorb onto foreign surfaces within the first few minutes of exposure, the 90 min incubation time allows fibrinogen more than enough time to settle onto the sponges.⁶⁷ After incubation and infinite dilution, PU-ZnO—Cu-PDMS-10 sponges were found to exhibit significant antifouling efficacy by reducing fibrinogen adhesion by 76.62±11.05% (p<0.01, n=6) when compared to control sponges (FIG. 8a). Additionally, to ensure that ZnO—Cu-PDMS coatings are also able to repel the cellular contents of blood, the control sponge and Pu-ZnO—Cu-PDMS-10 sponges were exposed to platelet-rich porcine plasma for 90 min under physiological conditions. Once again, Pu-ZnO—Cu-PDMS-10 sponges showed significant antifouling efficacy by reducing platelet adhesion by 64.16±11.42% (p<0.05, n=6) relative to control sponges (FIG. 8b) using a LDH assay. These results are in good agreement with the previous studies showing that superhydrophobic surfaces could significantly reduce both protein and platelet adhesion.^{35,36,68,69} On the other hand, similar to bacterial attachment, some of the superhydrophobic surfaces fail to resist protein and platelet adhesion,^{68,76} demonstrating poor antifouling efficacy in comparison to the fabricated sponges. Therefore, by exhibiting antifouling efficacy against both fibrinogen and platelet cells, it can be concluded that ZnO—Cu-PDMS-coated sponges may be used in blood-related applications.

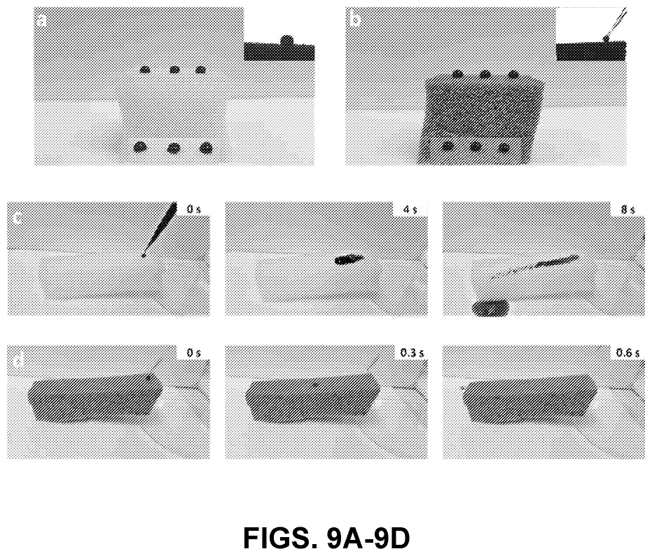

The superhydrophobic PU-ZnO—Cu-PDMS-10 sponges also exhibited high CAs with low adhesion to human blood. The CA of a 10 μL blood droplet on the sponge was 165.4±0.9° (FIG. 9b and FIG. 22) relative to 106.4±8.9° on the original surface (FIG. 9a and FIG. 22). FIGS. 9c and 22 demonstrate that while the surface of the bare sponge got contaminated by blood droplets, leaving a large blood trail along its travelled path, they could easily slide on the superhydrophobic sponge without leaving any visible trace behind (FIG. 9d).

Robustness of Coatings. In real-world applications, superhydrophobic coatings usually depend on a fragile micro/ nanostructure for their excellent water-repellency and are hence prone to wear by abrasion, resulting in the loss of surface superhydrophobicity. Therefore, it is essential to examine the mechanical stability of the fabricated superhydrophobic coatings. Different methods have been carried out to evaluate the mechanical properties of the PU-ZnO—Cu-PDMS-10 sample. (i) Scotch tape, finger-wipe, bending, and compression-release tests as well as various man-made destruction tests (e.g., single-hand-grasp, and both-hand-kneading tests) were applied to characterize adhesion of the particles to the underlying substrate; (ii) the surfaces were scratched (horizontal and vertical cuts) using a scalpel, and the sand paper test was applied to show the resistance of the coatings to massive mechanical damage.

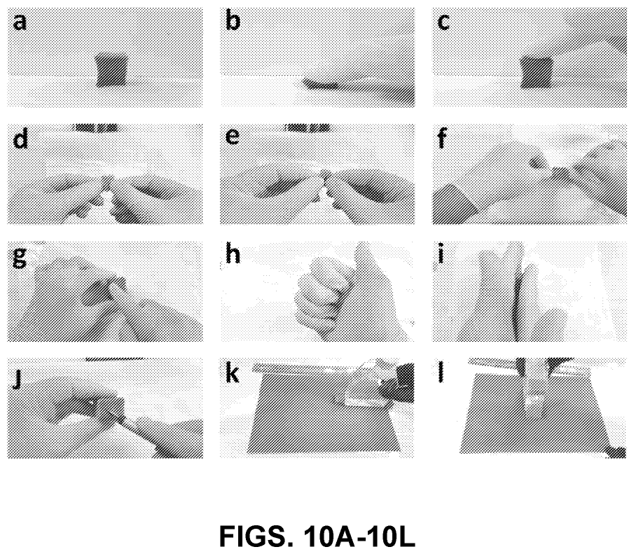

FIGS. 10a-c show that PU-ZnO—Cu-PDMS-10 was able to sustain its superhydrophobicity and original shape without detaching any nanoparticle fragments even after 1000 cycles of compression and release, indicating outstanding flexibility and mechanical robustness. The sponge was also repeatedly bent forward and backward, from −90 to 90 (defined as 1 cycle) over 50 folding cycles (FIG. 10d). Any delamination, fracturing, cracking, or peeling of the coating was not observed even after 50 repeated bending cycles, and the coating maintained its extreme water repellency unchanged and water droplets rolled away easily. Moreover, the superhydrophobicity of the sponge remain unchanged after 50 peeling cycles, which suggests that the particles are strongly anchored on the sponge skeleton (FIG. 10f). As demonstrated in FIG. 10g-i, the mechanical stability of the coatings was further investigated using various types of man-made destructions including finger-wipe, single-hand-grasp and both-hand-kneading tests. The results indicated that the sponge sustained its water-repellent property and the methylene blue-labeled water droplets could still readily roll off from the sponge surface after each test. The strong adhesion of the coating could also be reflected through the knife scratch and sandpaper abrasion tests. Upon being scratched with a knife, the exposed surfaces retained their superhydrophobicity and water droplets could easily roll off the damaged surface without leaving any traces behind (FIG. 10j), which suggests that the sponge is superhydrophobic throughout its whole volume.

The sandpaper abrasion test was performed using 400 grit SiC sandpaper as an abrasion surface. The PU-ZnO—Cu-PDMS-10 sample with a weight of 250 g was put above it face-down to sandpaper and was rubbed longitudinally and transversely (over 1000 cm in total). FIGS. 10k,l show that the surface of the sponge maintained its superhydrophobicity even after 1000 cm of sand abrasion, indicating its high tolerance upon mechanical damage. The results suggest that PU, an elastic polymer, could be used as a wear-resistant material in order to provide physical support to the incorporated particles, protecting them from being worn out. The microstructures on the elastic PU surface can be compressed to avoid being broken. The deformation will recover to its original structures when the external force is removed and helps stabilize the air cushions trapped in the microstructures that are essential to sustain the durable superhydrophobic surfaces. Therefore, the superhydrophobic surface shows outstanding mechanical durability.

### Conclusions

In summary, a facile method to fabricate flexible and mechanically durable superhydrophobic PU sponge materials were provided. The results show that the combination of liquid repellency and antibacterial NPs endows the sponges with antiadhesive properties that not only repel various liquids including whole blood but also resist adhesion of blood components and bacteria in addition to showing potent bactericidal activity without being toxic to mammalian cells. Importantly, by taking the advantages of PU with outstanding flexibility and high porosity, the coated sponges retained their antiwetting behavior even after harsh durability tests, hence showing their outstanding robustness.

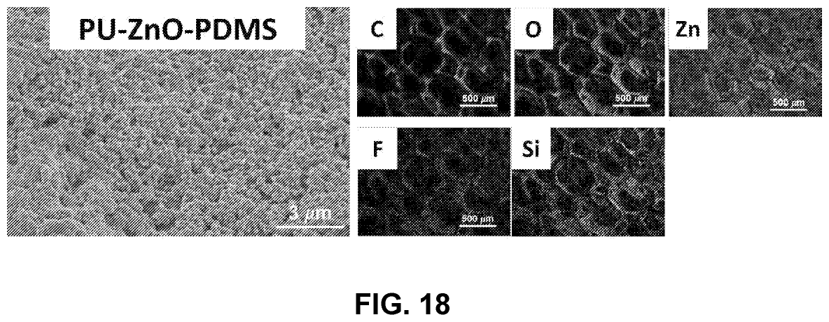

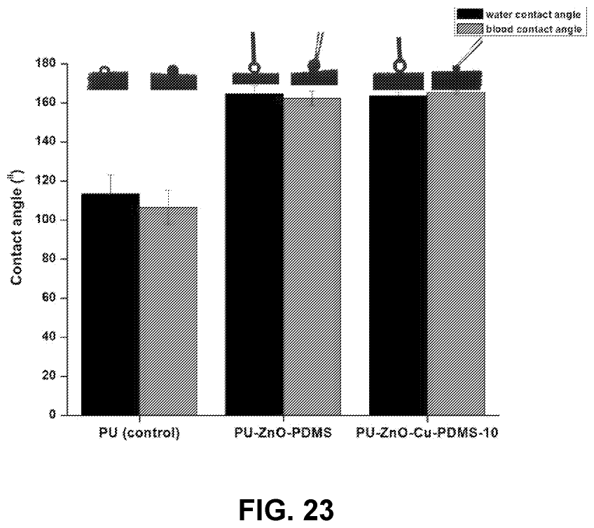

Supporting Information: The surface morphology of the as-prepared particles was characterized in detail. FIGS. 11-23 provide additional information. From the TEM and SEM images in FIG. 14, one can observe that there was no notable difference between the structures and sizes of the particles before and after the flourionization process. In general, the bare ZnO (43±24 nm) and Cu nanoparticles (44±16 nm) (FIG. 15) were irregular hexagonal and spherical in shape, respectively and tend to aggregate leading to the formation of larger clusters. For Cu-doped ZnO powders, they exhibit similar irregular morphology in which Cu NPs are distributed within and on the ZnO NP host, forming the hierarchical porous structures with micro/nano-roughness features that is vital in achieving liquid-repellent properties. The EDS mapping images (FIG. 14) also show that the main composition of the functionalized particles are zinc, oxygen, and copper from ZnO and Cu NPs, respectively and the distribution of Cu increases with an increased Cu concentration in the paints. Moreover, FAS modification resulted in appearance of the elements F and small amounts of Si, coming from FAS-17. FIG. 14 also demonstrates the XRD patterns of the particles, which reveals that all the particles are highly crystalline in nature. Also, the XRD pattern of the bare ZnO and FAS-ZnO NPs match well with that of hexagonal wurtzite ZnO structure, confirming that the FAS-treatment does not change the internal structure of the particles. For the ZnO/Cu samples, the diffraction patterns were similar to those of pure ZnO and Cu NPs, providing further evidence that the composite particles were composed of hexagonal ZnO, cubic Cu and Cu₂O nanocrystals.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES

Brown, M. R. W.; Allison, D. G.; Gilbert, P. Resistance of Bacterial Biofilms to Antibiotics a Growth-Rate Related Effect? J. Antimicrob. Chemother. 1988, 22, 777-780.
Costerton, J. W.; Lewandowski, Z.; Caldwell, D. E.; Korber, D. R.; Lappin-Scott, H. M. Microbial Biofilms. Annu. Rev. Microbiol. 1995, 49, 711-745.
Davies, D. Understanding Biofilm Resistance to Antibacterial Agents. Nat. Rev. Drug Discovery 2003, 2, 114-122.
Costerton, J. W.; Cheng, K. J.; Geesey, G. G.; Ladd, T. I.; Nickel, J. C.; Dasgupta, M.; Marrie, T. J. Bacterial Biofilms in Nature and Disease. Annu. Rev. MicrobioL 1987, 41, 435-464.
Anderson, R. L.; Holland, B. W.; Carr, J. K.; Bond, W. W.; Favero, M. S. Effect of Disinfectants on Pseudomonads Colonized on the Interior Surface of PVC Pipes. Am. J. Publ. Health 1990, 80, 17-21.
Szewzyk, U.; Szewzyk, R.; Manz, W.; Schleifer, K.-H. Microbiological Safety of Drinking Water. Annu. Rev. Microbiol. 2000, 54, 81-127.
Ling, F.; Whitaker, R.; LeChevallier, M. W.; Liu, W.-T. Drinking Water Microbiome Assembly Induced by Water Stagnation. ISME J. 2018, 12, 1520-1531.
Nemati, M.; Jenneman, G. E.; Voordouw, G. Mechanistic Study of Microbial Control of Hydrogen Sulfide Production in Oil Reservoirs. Biotechnol. Bioeng. 2001, 74, 424-434.
Trautner, B. W.; Darouiche, R. O. Role of Biofilm in Catheter-Associated Urinary Tract Infection4. Am. J. Infect. Contr. 2004, 32, 177-183.
Tiller, J. C.; Liao, C.-J.; Lewis, K.; Klibanov, A. M. Designing Surfaces That Kill Bacteria on Contact. Proc. Natl. Acad. Sci. U.S.A. 2001, 98, 5981-5985.
Ivanova, E. P.; Hasan, J.; Webb, H. K.; Truong, V. K.; Watson, G. S.; Watson, J. A.; Baulin, V. A.; Pogodin, S.; Wang, J. Y.; Tobin, M. J.; Löbbe, C.; Crawford, R. J. Natural Bactericidal Surfaces: Mechanical Rupture of Pseudomonas Aeruginosa Cells by Cicada Wings. Small 2012, 8, 2489-2494.
Landini, P.; Antoniani, D.; Burgess, J. G.; Nijland, R. Molecular Mechanisms of Compounds Affecting Bacterial Biofilm Formation and Dispersal. Appl. Microbiol. Biotechnol. 2010, 86, 813-823.
Liu, R.; Liu, X.; Zhou, J.; Nie, Q.; Meng, J.; Lin, J.; Wang, S. Bioinspired Superhydrophobic Ni—Ti Archwires with Resistance to Bacterial Adhesion and Nickel Ion Release. Adv. Mater. Interfaces 2019, 6, 1801569.
Wang, Z.; Su, Y.; Li, Q.; Liu, Y.; She, Z.; Chen, F.; Li, L.; Zhang, X.; Zhang, P. Researching a Highly Anti-Corrosion Super-hydrophobic Film Fabricated on AZ91D Magnesium Alloy and Its Anti-Bacteria Adhesion Effect. Mater. Charact. 2015, 99, 200-209.
Genzer, J.; Efimenko, K. Recent Developments in Super-hydrophobic Surfaces and Their Relevance to Marine Fouling: A Review. Biofouling 2006, 22, 339-360.
Ellinas, K.; Kefallinou, D.; Stamatakis, K.; Gogolides, E.; Tserepi, A. Is There a Threshold in the Antibacterial Action of Superhydrophobic Surfaces? ACS Appl. Mater. Interfaces 2017, 9, 39781-39789.
Li, M.; Gao, L.; Schlaich, C.; Zhang, J.; Donskyi, I. S.; Yu, G.; Li, W.; Tu, Z.; Rolff, J.; Schwerdtle, T.; Haag, R.; Ma, N. Construction of Functional Coatings with Durable and Broad-Spectrum Anti-bacterial Potential Based on Mussel-Inspired Dendritic Polyglycerol and in Situ-Formed Copper Nanoparticles. ACS Appl. Mater. Interfaces 2017, 9, 35411-35418.
Sehmi, S. K.; Noimark, S.; Pike, S. D.; Bear, J. C.; Peveler, W. J.; Williams, C. K.; Shaffer, M. S. P.; Allan, E.; Parkin, I. P.; MacRobert, A. J. Enhancing the Antibacterial Activity of Light-Activated Surfaces Containing Crystal Violet and ZnO Nanoparticles: Investigation of Nanoparticle Size, Capping Ligand, and Dopants. ACS Omega 2016, 1, 334-343.
Ozkan, E.; Crick, C. C.; Taylor, A.; Allan, E.; Parkin, I. P. Copper-Based Water Repellent and Antibacterial Coatings by Aerosol Assisted Chemical Vapour Deposition. Chem. Sci. 2016, 7, 5126-5131.
Ozkan, E.; Allan, E.; Parkin, I. P. White-Light-Activated Antibacterial Surfaces Generated by Synergy between Zinc Oxide Nanoparticles and Crystal Violet. ACS Omega 2018, 3, 3190-3199.
Ozkan, E.; Ozkan, F. T.; Allan, E.; Parkin, I. P. The Use of Zinc Oxide Nanoparticles to Enhance the Antibacterial Properties of Light-Activated Polydimethylsiloxane Containing Crystal Violet. RSC Adv. 2014, 5, 8806.
Leslie, D. C.; Waterhouse, A.; Berthet, J. B.; Valentin, T. M.; Watters, A. L.; Jain, A.; Kim, P.; Hatton, B. D.; Nedder, A.; Donovan, K.; Super, E. H.; Howell, C.; Johnson, C. P.; Vu, T. L.; Bolgen, D. E.; Rifai, S.; Hansen, A. R.; Aizenberg, M.; Super, M.; Aizenberg, J.; Ingber, D. E. A Bioinspired Omniphobic Surface Coating on Medical Devices Prevents Thrombosis and Biofouling. Nat. Biotechnol. 2014, 32, 1134-1140.
Smith, S. A.; Travers, R. J.; Morrissey, J. H. How It All Starts: Initiation of the Clotting Cascade. Crit. Rev. Biochem. Mol. Biol. 2015, 50, 326-336.
Palta, S.; Saroa, R.; Palta, A. Overview of the Coagulation System. Indian J. Anaesth. 2014, 58, 515.
Yun, S.-H.; Sim, E.-H.; Goh, R.-Y.; Park, J.-I.; Han, J.-Y. Platelet Activation: The Mechanisms and Potential Biomarkers. BioMed Res. Int. 2016, 2016, 1-5.
Cheung, P.-Y.; Sales, E.; Schulz, R.; Radomski, M. W. Nitric Oxide and Platelet Function: Implications for Neonatology. Semin. Perinatol. 1997, 21, 409-417.
Doolittle, R. F. The Structure and Evolution of Vertebrate Fibrinogen: A Comparison of the Lamprey and Mammalian Proteins. Adv. Exp. Med. Biol. 1990, 281, 25-37.
Kattula, S.; Byrnes, J. R.; Wolberg, A. S. Fibrinogen and Fibrin in Hemostasis and Thrombosis. Arterioscler., Thromb., Vasc. Biol. 2017, 37 ( ). DOI: DOI: 10.1161/ATVBAHA.117.308564.
Gorbet, M. B.; Sefton, M. V. Biomaterial-Associated Thrombosis: Roles of Coagulation Factors, Complement, Platelets and Leukocytes. Biomaterials 2004, 25, 5681-5703.
Stewart, P. S.; Franklin, M. J. Physiological Heterogeneity in Biofilms. Nat. Rev. Microbiol. 2008, 6, 199-210.
Bonifait, L.; Grignon, L.; Grenier, D. Fibrinogen InducesBiofilm Formation by Streptococcus Suis and Enhances Its Antibiotic Resistance. Appl. Environ. Microbiol. 2008, 74, 4969-4972.
Bittl, J. A. Coronary Stent Occlusion: Thrombus Horribilis. J. Am. Coll. Cardiol. 1996, 28, 368-370.
Silverstein, M. D.; Heit, J. A.; Mohr, D. N.; Petterson, T. M.; O'Fallon, W. M.; Melton, L. J. Trends in the Incidence of Deep Vein Thrombosis and Pulmonary Embolism. Arch. Intern. Med. 1998, 158, 585-593. Zhu, T.; Wu, J.; Zhao, N.; Cai, C.; Qian, Z.; Si, F.; Luo, H.; Guo, J.; Lai, X.; Shao, L.; Xu, J. Superhydrophobic/Superhydrophilic Janus Fabrics Reducing Blood Loss. Adv. Healthcare Mater. 2018, 7, 1701086.
Li, C.; Ye, W.; Jin, J.; Xu, X.; Liu, J.; Yin, J. Immobilization of Nattokinase-Loaded Red Blood Cells on the Surface of Super- hydrophobic Polypropylene Targeting Fibrinolytic Performance. J. Mater. Chem. B 2015, 3, 3922-3926.
Hoshian, S.; Kankuri, E.; Ras, R. H. A.; Franssila, S.; Jokinen, V. Water and Blood Repellent Flexible Tubes. Sci. Rep. 2017, 7, 1-8.
Moradi, S.; Hadjesfandiari, N.; Toosi, S. F.; Kizhakkedathu, J. N.; Hatzikiriakos, S. G. Effect of Extreme Wettability on Platelet Adhesion on Metallic Implants: From Superhydrophilicity to Superhydrophobicity. ACS Appl. Mater. Interfaces 2016, 8, 17631-17641.
Wan, P.; Wu, J.; Tan, L.; Zhang, B.; Yang, K. Research on Super-Hydrophobic Surface of Biodegradable Magnesium Alloys Used for Vascular Stents. Mater. Sci. Eng., C 2013, 33, 2885-2890.
Yang, Y.; Lai, Y.; Zhang, Q.; Wu, K.; Zhang, L.; Lin, C.; Tang, P. A Novel Electrochemical Strategy for Improving Blood Compatibility of Titanium-Based Biomaterials. Colloids Surf., B 2010, 79, 309-313.
Pant, J.; Goudie, M. J.; Hopkins, S. P.; Brisbois, E. J.; Handa, H. Tunable Nitric Oxide Release from S-Nitroso-N-Acetylpenicill-amine via Catalytic Copper Nanoparticles for Biomedical Applica-tions. ACS Appl. Mater. Interfaces 2017, 9, 15254-15264.
Singha, P.; Workman, C. D.; Pant, J.; Hopkins, S. P.; Handa, H. Zinc-Oxide Nanoparticles Act Catalytically and Synergistically with Nitric Oxide Donors to Enhance Antimicrobial Efficacy. J. Biomed. Mater. Res., Part A 2019, 107. DOI: DOI: 10.1002/jbm.a.36657.
Sivaraman, B.; Latour, R. A. The Relationship between Platelet Adhesion on Surfaces and the Structure versus the Amount of Adsorbed Fibrinogen. Biomaterials 2010, 31, 832-839.
Wang, B.; Zhang, Y.; Shi, L.; Li, J.; Guo, Z. Advances in the Theory of Superhydrophobic Surfaces. J. Mater. Chem. 2012, 22, 20112.
Brassard, J.-D.; Sarkar, D. K.; Perron, J.; Brassard, J.-D.; Sarkar, D. K.; Perron, J. Fluorine Based Superhydrophobic Coatings. Appl. Sci. 2012, 2, 453-464.
Ujjain, S. K.; Roy, P. K.; Kumar, S.; Singha, S.; Khare, K. Uniting Superhydrophobic, Superoleophobic and Lubricant Infused Slippery Behavior on Copper Oxide Nano-Structured Substrates. Sci. Rep. 2016, 6, 1-10.
Hackenberg, S.; Scherzed, A.; Technau, A.; Kessler, M.; Froelich, K.; Ginzkey, C.; Koehler, C.; Burghartz, M.; Hagen, R.; Kleinsasser, N. Cytotoxic, Genotoxic and pro-Inflammatory Effects of Zinc Oxide Nanoparticles in Human Nasal Mucosa Cells in Vitro. Toxicol. In Vitro 2011, 25, 657-663.
Bondarenko, O.; Juganson, K.; Ivask, A.; Kasemets, K.; Mortimer, M.; Kahru, A. Toxicity of Ag, CuO and ZnO Nanoparticles to Selected Environmentally Relevant Test Organisms and Mamma-lian Cells in Vitro: A Critical Review. Arch. Toxicol. 2013, 87, 1181-1200.
Saranya, S.; Vijayaranai, K.; Pavithra, S.; Raihana, N.; Kumanan, K. In vitro cytotoxicity of zinc oxide, iron oxide and copper nanopowders prepared by green synthesis. Toxicol. Rep. 2017, 4, 427-430.
Xu, P.; Li, Z.; Zhang, X.; Yang, Z. Increased Response to Oxidative Stress Challenge of Nano-Copper-Induced Apoptosis in Mesangial Cells. J. Nanopart. Res. 2014, 16, 1-13.
Zhang, C.-H.; Wang, Y.; Sun, Q.-Q.; Xia, L.-L.; Hu, J.-J.; Cheng, K.; Wang, X.; Fu, X.-X.; Gu, H. Copper Nanoparticles Show Obvious in Vitro and in Vivo Reproductive Toxicity via ERK Mediated Signaling Pathway in Female Mice. Int. J. Biol. Sci. 2018, 14, 1834-1844.
Gupta, A.; Das, R.; Yesilbag Tonga, G.; Mizuhara, T.; Rotello, V. M. Charge-Switchable Nanozymes for Bioorthogonal Imaging of Biofilm-Associated Infections. ACS Nano 2018, 12, 89-94.
Xu, C.; Jiang, J.; Oguzlu, H.; Zheng, Y.; Jiang, F. Antifouling, Antibacterial and Non-Cytotoxic Transparent Cellulose Membrane with Grafted Zwitterion and Quaternary Ammonium Copolymers. Carbohydr. Polym. 2020, 250, 116960.
Guo, L.; Panderi, I.; Yan, D. D.; Szulak, K.; Li, Y.; Chen, Y.-T.; Ma, H.; Niesen, D. B.; Seeram, N.; Ahmed, A.; Yan, B.; Pantazatos, D.; Lu, W. A Comparative Study of Hollow Copper Sulfide Nanoparticles and Hollow Gold Nanospheres on Degradability and Toxicity. ACS Nano 2013, 7, 8780-8793.
Xiao, J.; Chen, S.; Yi, J.; Zhang, H. F.; Ameer, G. A. A Cooperative Copper Metal-Organic Framework-Hydrogel System Improves Wound Healing in Diabetes. Adv. Funct. Mater 2017, 27, 1604872.
Xiao, J.; Zhu, Y.; Huddleston, S.; Li, P.; Xiao, B.; Farha, O. K.; Ameer, G. A. Copper Metal-Organic Framework Nanoparticles Stabilized with Folic Acid Improve Wound Healing in Diabetes. ACS Nano 2018, 12, 1023-1032.
Mroczek-Sosnowska, N.; Sawosz, E.; Vadalasetty, K.; Lukasiewicz, M.; Niemiec, J.; Wierzbicki, M.; Kutwin, M.; Jaworski, S.; Chwalibog, A. Nanoparticles of Copper Stimulate Angiogenesis at Systemic and Molecular Level. Int. J. Mol. Sci. 2015, 16, 4838-4849.
Zhao, S.; Li, L.; Wang, H.; Zhang, Y.; Cheng, X.; Zhou, N.; Rahaman, M. N.; Liu, Z.; Huang, W.; Zhang, C. Wound Dressings Composed of Copper-Doped Borate Bioactive Glass Microfibers Stimulate Angiogenesis and Heal Full-Thickness Skin Defects in a Rodent Model. Biomaterials 2015, 53, 379-391.
Cao, B.; Zheng, Y.; Xi, T.; Zhang, C.; Song, W.; Burugapalli, K.; Yang, H.; Ma, Y. Concentration-Dependent Cytotoxicity of Copper Ions on Mouse Fibroblasts in Vitro: Effects of Copper Ion Release from TCu380A vs TCu220C Intra-Uterine Devices. Biomed. Micro-devices 2012, 14, 709-720.
Rice, L. B. Challenges in Identifying New Antimicrobial Agents Effective for Treating Infections with Acinetobacter Baumannii and Pseudomonas Aeruginosa. Clin. Infect. Dis. 2006, 43, S100-S105.
Homeyer, K. H.; Goudie, M. J.; Singha, P.; Handa, H. Liquid- Infused Nitric-Oxide-Releasing Silicone Foley Urinary Catheters for Prevention of Catheter-Associated Urinary Tract Infections. ACS Biomater. Sci. Eng. 2019, 5, 2021-2029.
Sawant, S. N.; Selvaraj, V.; Prabhawathi, V.; Doble, M. Antibiofilm Properties of Silver and Gold Incorporated PU, PCLm, PC and PMMA Nanocomposites under Two Shear Conditions. PLoS One 2013, 8, No. e63311. Liu, J.; Ye, L.; Sun, Y.; Hu, M.; Chen, F.; Wegner, S.; Maila\ddot{n}der, V.; Steffen, W.; Kappl, M.; Butt, H. J. Elastic Super-hydrophobic and Photocatalytic Active Films Used as Blood Repellent Dressing. Adv. Mater 2020, 32, 1908008.
Ren, T.; Yang, M.; Wang, K.; Zhang, Y.; He, J. CuO Nanoparticles-Containing Highly Transparent and Superhydrophobic Coatings with Extremely Low Bacterial Adhesion and Excellent Bactericidal Property. ACS Appl. Mater. Interfaces 2018, 10, 25717-25725.
Hwang, G. B.; Page, K.; Patir, A.; Nair, S. P.; Allan, E.; Parkin, I. P. The Anti-Biofouling Properties of Superhydrophobic Surfaces Are Short-Lived. ACS Nano 2018, 12, 6050-6058.
Zhou, X.; Lee, Y.-Y.; Chong, K. S. L.; He, C. Superhydrophobic and Slippery Liquid-Infused Porous Surfaces Formed by the Self-Assembly of a Hybrid ABC Triblock Copolymer and Their Antifouling Performance. J. Mater. Chem. B 2018, 6, 440-448.
Werner, C.; Maitz, M. F.; Sperling, C. Current Strategies towards Hemocompatible Coatings. J. Mater. Chem. 2007, 17, 3376.
Statz, A. R.; Barron, A. E.; Messersmith, P. B. Protein, Cell and Bacterial Fouling Resistance of Polypeptoid-Modified Surfaces: Effect of Side-Chain Chemistry. Soft Matter 2008, 4, 131-139.
Movafaghi, S.; Leszczak, V.; Wang, W.; Sorkin, J. A.; Dasi, L. P.; Popat, K. C.; Kota, A. K. Hemocompatibility of Superhemophobic Titania Surfaces. Adv. Healthcare Mater 2017, 6, 1600717.
Bartlet, K.; Movafaghi, S.; Kota, A.; Popat, K. C. Super-hemophobic Titania Nanotube Array Surfaces for Blood Contacting Medical Devices. RSC Adv. 2017, 7, 35466-35476.
Koc, Y.; De Mello, A. J.; McHale, G.; Newton, M. I.; Roach, P.; Shirtcliffe, N. J. Nano-Scale Superhydrophobicity: Suppression of Protein Adsorption and Promotion of Flow-Induced Detachment. Lab Chip 2008, 8, 582-586.

1. A coating composition comprising
(a) zinc oxide nanoparticles:
(b) copper nanoparticles;
(c) a perfluorolkylsiloxane: and
(d) an organic solvent.

2. The composition of claim 1, wherein the zinc oxide nanoparticles have an average particle size of from about 20 nm to about 70 nm.

3. The composition of claim 1, wherein the zinc oxide nanoparticles are from about 1% to about 20% by weight of the composition.

4. The composition of claim 1, wherein the copper nanoparticles have an average particle size of from about 20 nm to about 70 nm.

5. The composition of claim 1, wherein the copper nanoparticles are from about 0.1% to about 5% by weight of the composition.

6. The composition of claim 1, wherein the perfluorolkylsiloxane has the formula R²—Si(OR¹)₃, wherein R¹ is a substituted or unsubstituted C₁-C₂₀ alkyl group, and R² is a C₁-C₂₀ perfluoroalkyl group.

7. The composition of claim 6, wherein R¹ is a C₁ to C₄ alkyl group.

8. The composition of claim 6, wherein R² is a C₁ to C₁₀ perfluoroalkyl group.

9. The composition of claim 6, wherein each R¹ is methyl or ethyl, and R² is a C₈ perfluoroalkyl group.

10. The composition of claim 1, wherein the perfluorolkylsiloxane is from about 0.1% to about 2% by weight of the composition.

11. The composition of claim 1, wherein the organic solvent comprises an alcohol or a hydrocarbon.

12. The composition of claim 1, wherein the organic solvent comprises a C₁ to C₁₀ alcohol.

13. The composition of claim 1, wherein the organic solvent comprises methanol, ethanol, propanol, isopropanol, butanol, or any combination thereof.

14. The composition of claim 1, wherein the organic solvent is from about 73% to about 98.8% by weight of the composition.

15. The composition of claim 1, wherein the composition is produced by mixing the zinc oxide nanoparticles, the copper nanoparticles, and the perfluorolkylsiloxane in the organic solvent.

16. The composition of claim 1, wherein the perfluorolkylsiloxane is covalently bonded to the zinc oxide nanoparticles, the copper nanoparticles, or a combination thereof.

17. The composition of claim 1, wherein the weight ratio of the zinc oxide nanoparticles to the copper nanoparticles is from 1:1 to 20:1.

18. The composition of claim 1, wherein the weight ratio of the zinc oxide nanoparticles to the perfluoroalkysiloxane is from 5:1 to 20:1

19. The composition of claim 1, wherein the weight ratio of the copper nanoparticles to the perfluoroalkysiloxane is from 0.5:1 to 5:1

20. A coated article produced by the method comprising (a) applying the coating composition of claim 1 to at least one surface of the article and (b) removing the organic solvent from the coating composition to produce the coated article.

21. The coated article of claim 20, wherein the article is dipped into the coating composition.

22. The coated article of claim 20, wherein the coating composition is sprayed on at least one surface of the article.

23. The coated article of claim 20, wherein the organic solvent is removed by evaporation.

24. The coated article of claim 20, wherein step (b) comprises heating the coated article at a temperature of from about 80° C. to about 120° C. to remove the organic solvent.

25. The coated article of claim 20, wherein after step (b), applying to the coated article a polysiloxane.

26. The coated article of claim 25, wherein the polysiloxane comprises a polydimethylsiloxane, a polydiethylsiloxane, a polydipropylsiloxane, or a polydiphenylsiloxane.

27. A coated article comprising a first coating on at least one surface of the article, wherein the first coating comprises zinc oxide nanoparticles and copper nanoparticles covalently bonded to a perfluorolkylsiloxane.

28. The coated article of claim 27, wherein the coating composition further comprises Cu₂O.

29. The coated article of claim 27, wherein the article further comprises a second coating comprising a polysiloxane adjacent to the first coating.

30. The coated article of any one of claims 20 to 29, wherein the article comprises a polymeric grade material, a medical device, a surface or article in a hospital or medical facility, or a surface in an automobile, boat, or aircraft.

31. The coated article of any one of claims 20 to 29, wherein the article has a receding contact angle of from about 140 degrees to about 175 degrees.

32. The coated article of any one of claims 20 to 29, wherein the article has an advancing contact angle of from about 140 degrees to about 175 degrees.

33. The coated article of any one of claims 20 to 29, wherein the article has a contact angle hysteresis of from about 0.1 degrees to about 5 degrees.

34. The coated article of any one of claims 20 to 29, wherein the article has a static water contact angle of greater than 150 degrees.

35. The coated article of any one of claims 20 to 29, wherein the article maintains superhydrophobic properties when exposed to mechanical agitation.

36. The coated article of any one of claims 20 to 29, wherein the article is biocompatible.

37. A method for preventing the growth of bacteria on an article, the method comprising applying the coating composition of any one of claims 1 to 19 to at least one surface of the article.

38. A method for preventing the adhesion of fibrinogen on an article, the method comprising applying the coating composition of any one of claims 1 to 19 to at least one surface of the article.

39. A method for preventing the adhesion of platelets on an article, the method comprising applying the coating composition of any one of claims 1 to 19 to at least one surface of the article.
